(19) United States
(12) Patent Application Publication
(10) Pub. No.: US 2005/0287658 A1
(43) Pub. Date: Dec. 29, 2005

(54) Drainage bioremediation apparatuses and methods

(76) Inventor: Hansford R. Williams, Bradenton, FL (US)

Correspondence Address: LITMAN LAW OFFICES, LTD, PO BOX 15035, CRYSTAL CITY STATION, ARLINGTON, VA, 22215, US

(21) Appl. No.: 11/157,849

(22) Filed: Jun. 22, 2005

Related U.S. Application Data
(60) Provisional application No. 60/582,665, filed on Jun. 25, 2004.

(51) Int. Cl. C12S 9/00; C12M 1/36

(52) U.S. Cl. 435/264; 435/286.5; 435/289.1

(57) ABSTRACT

The method of the dual chamber bioremediation apparatus involves preparing a waste drain dosing batch of bioremedial bacteria in one chamber of the apparatus while dispensing another batch from a second chamber, and alternately switching the chamber functions. The enhanced replication bioremedial apparatus comprises a bioremedial pellet dispensing wheel, an aqueous replication chamber for growing bioremedial bacteria and a dispenser for recharging pellets and water to the chamber and bioremedial bacteria to a waste drain. The method of the invention involves preparing an initial amount of dosing mixture equal to twice the amount needed for a planned unit of dosing time. After dosing for unit time the chamber is recharged with water and pellets to the original volumn. The chamber bacterial residue stimulates replication of the added bacteria thereby producing highly efficacious levels of cell populations for bioremedial operations.

### CROSS-REFERENCE TO RELATED APPLICATION

This application claims the benefit of U.S. Provisional Patent Application Ser. No. 60/582,665, filed Jun. 25, 2004.

### BACKGROUND OF THE INVENTION

1 Field of the Invention

The present invention relates to apparatuses and methods for applying bioremediation bacteria to a drainage system. More specifically, the invention is directed to a dual chamber bioremediation apparatus and method for applying bioremediation bacteria on a continuous basis to a waste drain to break down grease or fat. In another aspect, the invention comprises an enhanced replication bioremediation apparatus and method (system 100) that applies high cell counts of bioremediation bacteria repeatedly and frequently to targeted destinations, typically a waste drain.

2. Description of the Related Art

Commercial and non-commercial operations engaged in mass preparation of food for human consumption generate a considerable amount of food waste, such as fats, grease, starches, and cellulose. Food waste is often mixed with water in a waste disposal unit operably attached to a sink. Sometimes hot water from a sink faucet is used as a carrier to dispose of fat and grease. Hot water may be mixed with unwanted grease and fat and the mixture is then directed to a drain for disposal. This is a common practice in food establishments, such as restaurants, bakeries, schools, prisons, resorts, hotels, distilleries, dairies, tanneries, breweries, ships (especially cruise ships), airports, delis, caterers, hospitals, vitamin manufacturers, butchers, canneries, etc. However, using hot water to dispose of fat and grease merely moves the problem downstream; when the hot water cools, the fat and grease drop out and build up to cause more serious problems, such as drain blockages, back-up, and objectionable odors. The hot water also causes problems because naturally occurring bacteria, which might breakdown the fat and grease to innocuous by-products, such as carbon dioxide and water, are killed upon contact with hot water, leaving fat and grease to build up. Using harsh chemicals, such as bleach, also harms the otherwise helpful, naturally occurring fat-digesting bacteria. Plumbing problems, such as back-ups, lead to additional costs, especially when intervention is required by, e.g., a plumbing firm.

Bioremediation is the term used to describe the use of microorganisms such as bacteria to break down undesirable substances, such as fats and grease. Left unchecked, grease build up in a drain line can lead to poor drainage issues and obnoxious smells emanating from the drain or waste line. Grease fouling of waste lines and drains is a particular problem in fast food restaurants that prepare and sell meat products.

U.S. Pat. No. 5,225,083, issued to Pappas et al. on Jul. 6, 1993, describes a method for bioremediation of grease traps. The '083 method comprises the steps of mechanically removing solid materials, such as plastic items, food particles and the like, from entrances to all drain lines and the drain lines themselves terminating in the grease traps; securing loose drain line covers and replacing broken covers; preventing the flow of all chemicals which are detrimental to the growth of endemic bacterial microorganisms into the drain lines and grease traps; adjusting the pH of the water effluent in the grease traps by introducing a basic material, such as baking soda, into the grease traps and mixing or stirring the water, which stimulates the endemic native bacteria resident in the grease trap; and adding endemic bacterial microorganisms to one or more of the drain lines for ultimate introduction into the grease traps and bio-digesting grease in the drain lines and grease traps. The '083 bioremediation method does not teach or suggest a continuous system of bacterial dosing based on the dual chamber bioremediation apparatus and method of the present invention.

U.S. Patent Publication No. 2002/0023875, published Feb. 28, 2002, describes a method and apparatus for clearing wastewater pipes and/or grease traps clogged with grease, with the method comprising the steps of: providing a dry agent comprising bacteria and enzymes; mixing the dry agent with an amount of water sufficient to cause in situ production of an aqueous mixture; maintaining the aqueous mixture in an activator vessel structure to activate the aqueous mixture for a time sufficient to form an aqueous solvent for cleaning or clearing fatty residues and/or grease; and contacting the wastewater pipes and/or grease traps containing fatty residues and/or grease with the aqueous solvent to dissolve the grease and/or fatty residues, thus cleaning the wastewater pipes and/or grease traps by bio-digesting fatty residues and/or grease deposited in the wastewater pipes and/or grease traps. The '875 publication does not teach or suggest a continuous system of bacterial dosing based on the dual chamber bioremediation apparatus and method of the present invention.

U.S. Pat. No. 3,242,055, issued to S. De Lucia on Mar. 22, 1966, describes a process and composition for enhancing bacterial action in wastewater by providing an enzyme pellet construction configured to dissolve at variable rates in order to provide immediate and lasting bacteria action. The '055 process and composition does not teach or suggest a continuous system of bacterial dosing based on the dual chamber bioremediation apparatus and method of the present invention.

U.S. Pat. No. 6,335,191, issued to Kiplinger et al. on Jan. 1, 2002, describes an automated system and method for cultivating bacteria in a fluid medium and thereafter selectively discharging the fluid medium, wherein an initial supply of the selected strain or strains of bacteria is combined with nutrients and water in a biogenerator in the presence of air to promote mixing and bacterial cultivation. The system and method utilize a vortex created by recirculation of the fluid medium to achieve aeration and mixing without substantial foaming. The system and method are particularly useful for supplying bacteria to control grease accumulation in restaurant grease traps. The system and method use a biogeneration chamber that has a cylindrical sidewall and surface on the inner side. Further, the chamber has a top and a conical bottom. The top has inlet ports and a vent port. There is also an outlet port in the conical bottom. The conical bottom also has an orifice and recirculated fluid inlet port that is directed tangentially along the inside surface of the sidewall to create a downwardly spiraling vortex in the biogeneration chamber. The '191 system and method does not teach or suggest a continuous system of bacterial dosing based on the dual chamber bioremediation apparatus and method of the present invention.

BESTechnologies, Inc. of Sarasota, Fla. developed a consortium of multiple strains of bioremediation bacteria for clearing fats and greases from drains in conjunction with the lab at Iowa State University. BESTechnologies marketed the consortium, at least as of March 2003, in the form of a freeze-dried powder. The bacteria are delivered using what is referred to as “The Bladder Bag System.” The customer is furnished with enough powder for a thirty-day supply. The powder is mixed with five gallons of water in a bag to activate the bacteria and form a “dosing material.” The dosing material is delivered from the bag to the drain by a peristaltic pump and control circuit that draws a small amount of the dosing material every two hours and dispenses it through a small, flexible plastic tube into the drain line that typically leads to a grease trap.

While effective in keeping drain lines clear, there are some problems with the bladder bag system. Servicing the system is inconvenient and time consuming. The bag must be changed every thirty days. Using the mixture requires the addition of five gallons of water (approximately forty pounds). Moreover, the bacteria colony count maximizes within the first day or so and then steadily diminishes throughout the monthly cycle. Consequently, there is a need for an apparatus and system that will dispense a dosing material of bioremediation bacteria while the colony count is still relatively high. There is a further need for a device that will mix and dispense the dosing material with a minimum amount of time and effort for maintenance.

None of the above inventions and patents, taken either singly or in combination, is seen to describe the instant invention as claimed. Thus, a continuous system of bacterial dosing based on the dual chamber bioremediation apparatus and method or the enhanced replication bioremediation apparatus and method of the present invention that solves the aforementioned problems is desired.

### SUMMARY OF THE INVENTION

(i) The dual chamber bioremediation apparatus applies bioremediation bacteria to a waste drain on a continuous basis. The apparatus operates upon a medium that includes a consortium or mixture of multiple strains of bioremediation bacteria together with appropriate nutrients that has been compressed into pellet form. A thirty-day supply of pellets is mounted in a cartridge and loaded into a pellet dispenser. A pan or other container with a dividing wall defines first and second chambers. Sufficient water for preparing a twenty-four hour supply is drawn into the first chamber and a pellet is dispensed into the first chamber. The dosing material in the first chamber is allowed to germinate for twenty-four hours to form a dosing material. A measured quantity of the dosing material is dispensed by a peristaltic pump at predetermined time intervals, such as every two hours, throughout the succeeding twenty-four hour period. Simultaneously, a second pellet is dropped into the second chamber with sufficient water for a twenty-four hour supply of dosing material an allowed to germinate. Thereafter the first and second chambers alternate between germinating a batch of dosing material and dispensing a batch of dosing material. The cartridge is replaced monthly to maintain the supply of pellets.

The method for applying continuous bioremediation bacteria to a waste drain comprises the steps of: (a) providing a dosing material of active bioremediation bacteria in a first chamber; (b) dispensing active bioremediation bacteria from the first chamber into a drain line while simultaneously preparing a dosing material of active bioremediation bacteria in a second chamber; and (c) dosing the drain line with active bioremediation bacteria from the second chamber in response to exhaustion of the active bioremediation bacteria in the first chamber, and upon exhaustion of the active bioremediation bacteria in the second chamber repeating steps (a), (b) and (c). In this manner, a continuous supply of active bioremediation bacteria is supplied to a drain line in sufficient numbers to keep the drain lines free of fats, grease, and other biological waste.

(ii) The enhanced replication bioremediation apparatus and method (system 100) of the invention applies high cell counts of bioremediation bacteria repeatedly and frequently to targeted destinations, typically a waste drain. Enhanced replication means that the method and apparatus of the invention substantially increases the rate of bioremediation bacteria cell division over bioremedial processes known in the art. The resulting increased population of bioremedial bacteria cells is regularly delivered in measured doses to a targeted destination to free that destination of offending organic matter.

The system 100 operates using a medium that includes a mixture or consortium of multiple strains of bioremediation bacteria together with appropriate nutrient, a binding agent and other lesser ingredients that have been compressed into a pellet. The dispenser pellets or medium may be obtained from Best Technologies, Inc., 7329 International Place, Sarasota, Fla. 34240 USA. A supply of pellets, typically enough to last for one month, is loaded and sealed into a cartridge and placed into the dispenser. A replication chamber located below the pellet cartridge has sufficient capacity to hold a two-day supply of dosing material. On day one of the monthly service cycle two pellets are dropped from the pellet wheel through a tower on the replication chamber cover and into a replication chamber below. A forty-eight hour supply of water is also drawn into the replication chamber. The replication process begins immediately, although it takes a day or so for the cell count of the bioremediation bacteria to replicate to near maximum concentration. A measured quantity of the resulting dosing material is dispensed by a peristaltic pump or other suitable pump at predetermined time intervals, typically every two hours throughout the succeeding twenty-four hours. At the end of each 24-hour period after refilling the replication chamber, approximately half of the dosing material will have been dispensed from the replication chamber. At this time, and repeating every 24 hours thereafter through the service cycle, typically 31 days, with the replication chamber approximately half-full of dosing material, an additional pellet and enough water is added to bring the level of dosing material in the replication chamber to full. It has been discovered and is most notable that by controlling dispensing events so that a meaningful amount of dosing material, e.g., preferably about one-half or between one-third and two-thirds, remains in the replication chamber when another pellet and additional water is added, the remaining dosing material serves as seed bacteria which enhances the replication process. The result is a much higher concentration of bioremediation bacteria than would be the case if the replication chamber were to be emptied completely before the addition of a pellet and water.

Given time, bioremediation bacteria have a tendency to mutate. However, because of the dilution that takes place when water is added as well as the addition of a new pellet containing the original strains each day, the bioremediation bacteria strains within the replication chamber remain relatively the same throughout the service cycle. When monthly service is performed, dosing material remaining in the replication chamber is removed and a new cycle begins, starting with a 48-hour supply of water and two new pellets. This insures that desirable strains continue to be the dominant strains indefinitely. The cartridge is typically replaced monthly to maintain the necessary supply of pellets.

The method for applying bioremediation bacteria repeatedly and frequently to a waste drain comprises the steps of: (a) maintaining a dosing material by drawing the dosing material down to about half empty before adding a pellet containing additional bioremediation bacteria and nutrient and refilling the chamber to full by adding water. In this manner, a supply of active bioremediation bacteria is dispensed repeatedly and frequently to a drain line in sufficient numbers to keep the drain line free of waste products.

These and other features of the present invention will be readily apparent upon consideration of the following specification and drawings.

### BRIEF DESCRIPTION OF THE DRAWINGS

FIGS. 1 through 11 relate to the embodiment of the dual chamber bioremediation apparatus and method of the invention. FIGS. 12-28 relate to the embodiment of the enhanced replication and bioremediation apparatus and method of the invention.

FIG. 1 is an environmental, perspective view of a dual chamber bioremediation apparatus according to the present invention.

FIG. 2 is a front view of a dual chamber bioremediation apparatus according to the present invention with the housing broken away and partially in section.

FIG. 3 is a block diagram of the electronic control circuitry of the dual chamber bioremediation apparatus according to the present invention.

FIG. 4 is a top view of a dual chamber bioremediation apparatus according to the present invention with the housing cover removed.

FIG. 5 is a section view drawn along lines 5-5 of FIG. 4.

FIG. 6 is a chart representing the average cell count of bacteria grown in a bladder bag system of the prior art from days 1-9.

FIG. 7 is a chart representing the average cell count of bacteria grown in a bladder bag system of the prior art from days 10-17.

FIG. 8 is a chart representing the average cell count of bacteria grown in a bladder bag system of the prior art from days 18-25.

FIG. 9 is a chart representing the average cell count of bacteria grown in a bladder bag system of the prior art from days 26-30.

FIG. 10 is a chart representing the average cell count of bacteria grown in the dual chamber bioremediation apparatus of the present invention from days 1-30.

FIG. 11 is a chart representing the average cell counts of bacteria grown in the dual chamber bioremediation apparatus of the present invention from day's 1-30 representing growth in all three-test vessels.

—Enhanced Replication Bioremediation Apparatuses and Method

FIG. 12 is an environmental perspective view of a battery powered enhanced replication chamber bioremediation apparatus and method according to the present invention, using a 5-gallon reservoir as the water source.

FIG. 13 is an environmental perspective view of a transformer powered enhanced replication chamber bioremediation apparatus and method according to the present invention using a pressurized water line as the water source.

FIG. 14 is a block diagram of the electrical system for an enhanced replication bioremediation apparatus and method according to the invention

FIG. 15 is an exploded perspective of a battery-powered dispenser 200 (reservoir not shown) as viewed from the left front.

FIG. 16 is an exploded perspective of a transformer powered dispenser 200, plumbed to a pressurized water line (pressurized water line not shown), as viewed from the left rear.

FIG. 17 is a perspective of the control module with the face of the pellet wheel, the pump cover and the right pump omitted.

FIG. 18 shows a modified block diagram of the plumbing layout.

FIG. 19 through 28, inclusive (ten figures) illustrate in drawing form the 10 ways the invention can be configured to meet the specific needs of the end-user.

Similar reference characters denote corresponding features consistently throughout the attached drawings.

### DETAILED DESCRIPTION OF THE PREFERRED EMBODIMENTS

1. The Dual Chamber Bioremediation Apparatus and Method

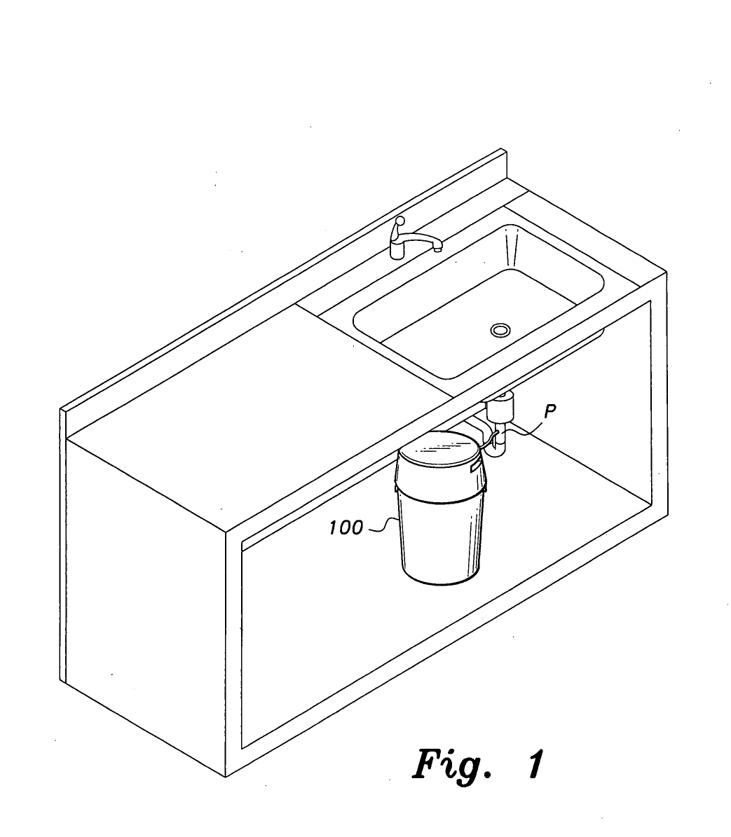

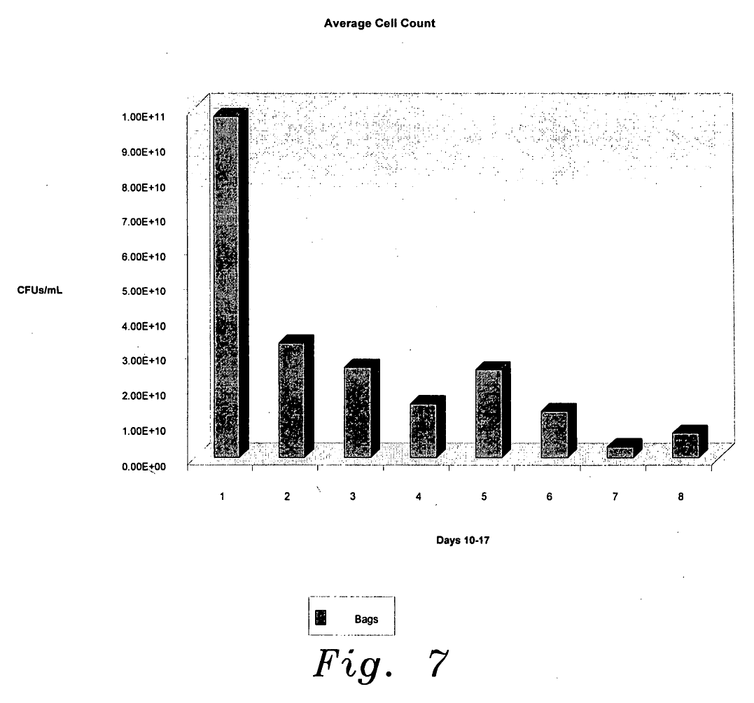

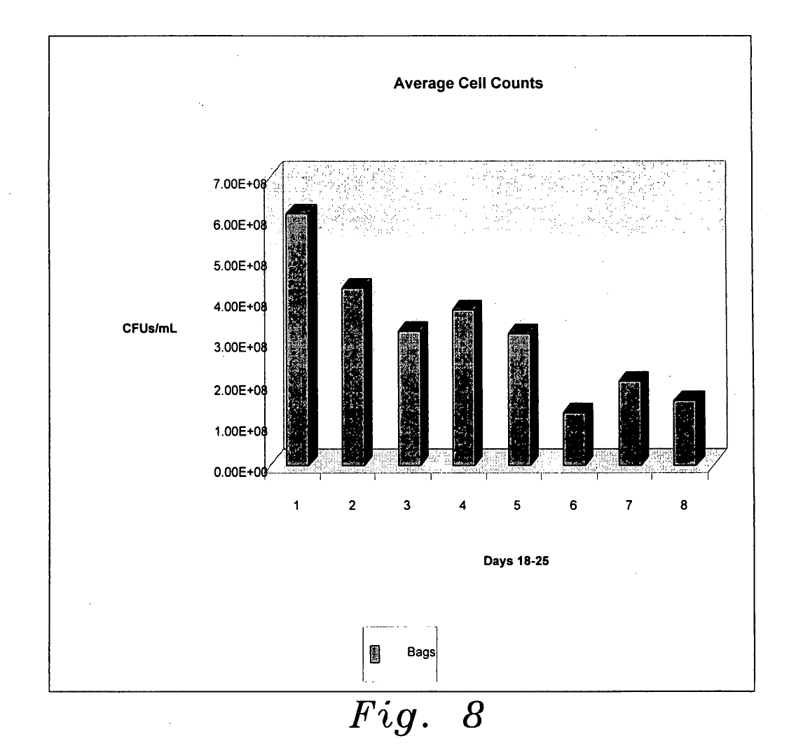

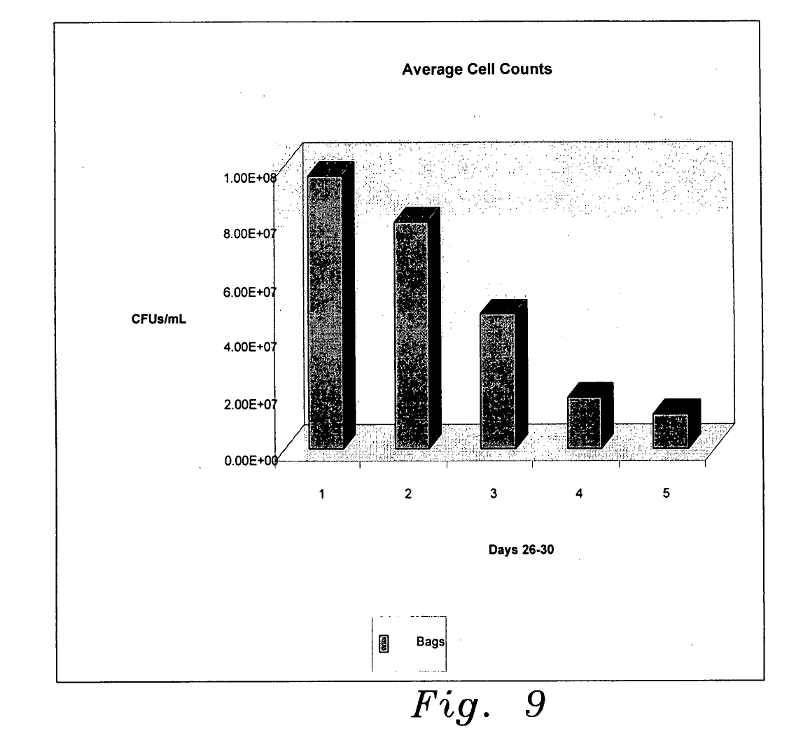

FIGS. 1-11:

The present invention is directed to an apparatus and method for applying bioremediation bacteria to a drainage system. More specifically, the invention is directed to an apparatus and method for applying bioremediation bacteria on a continuous basis to a waste drain to break down grease and/or fat. The apparatus is a dual chamber media generator and dispensing system, designated as 100 in the drawings. FIG. 1 shows the system 100 being used under a kitchen sink connected to a waste drain such as pipes P.

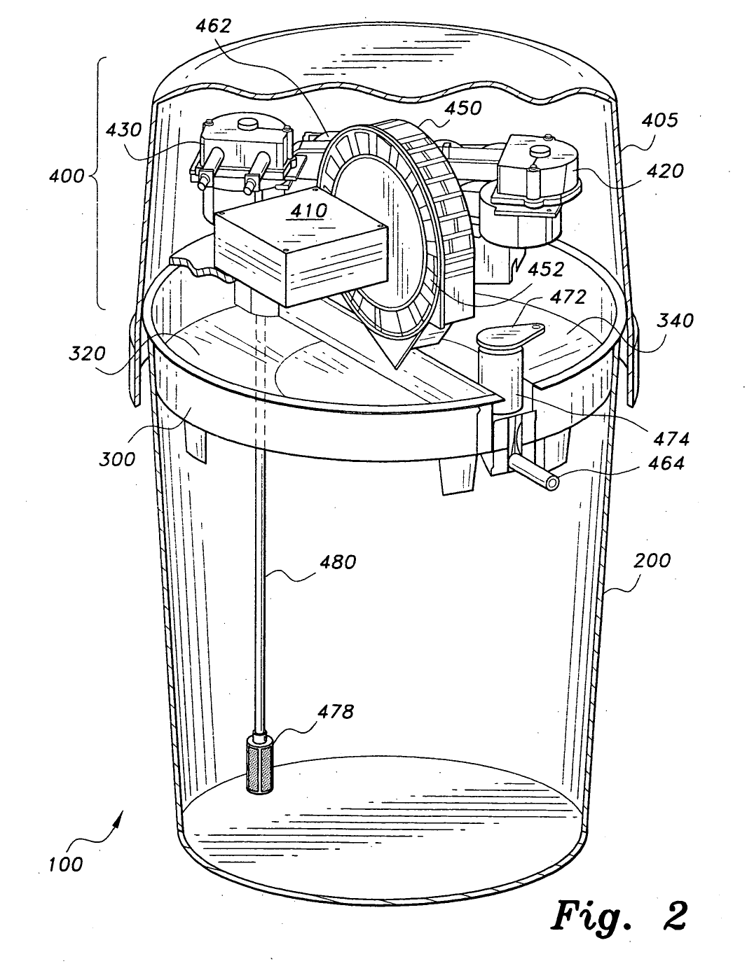

Referring now to FIG. 2, the apparatus 100 comprises a pail 200, a preparation container 300 and a dispenser device 400. The top of the pail 200 holds and supports the container 300. The dispenser 400 rests on top of the container 300 and sandwiches the container 300 between itself and the pail 200. The pail 200 is a five-gallon bucket, which serves as a water reservoir for the dispenser 400. However, the pail 200 is not essential to the apparatus 100, and the dispenser 400 may draw water from any other water source, e.g., pressurized tap water, or any other water reservoir.

The preparation container 300 is a dual chambered pan or other container having a partition or dividing wall defining a first chamber 320 and a second chamber 340 for selectively preparing or holding and dispensing a bioremediation dosing mixture. Each chamber 320, 340 is generally about half the diameter of the pail 200 and about 1½″ deep, preferably holding about 610 ml of water or dosing material. The dual chambers 320, 340 permit the preparation of the dosing material and the dispensation of the dosing material at various times to occur simultaneously. Thus, when one chamber is dispensing the dosing mixture into the waste drain, the other chamber is receiving a pellet and water from the water source and preparing the dosing mixture for the next day.

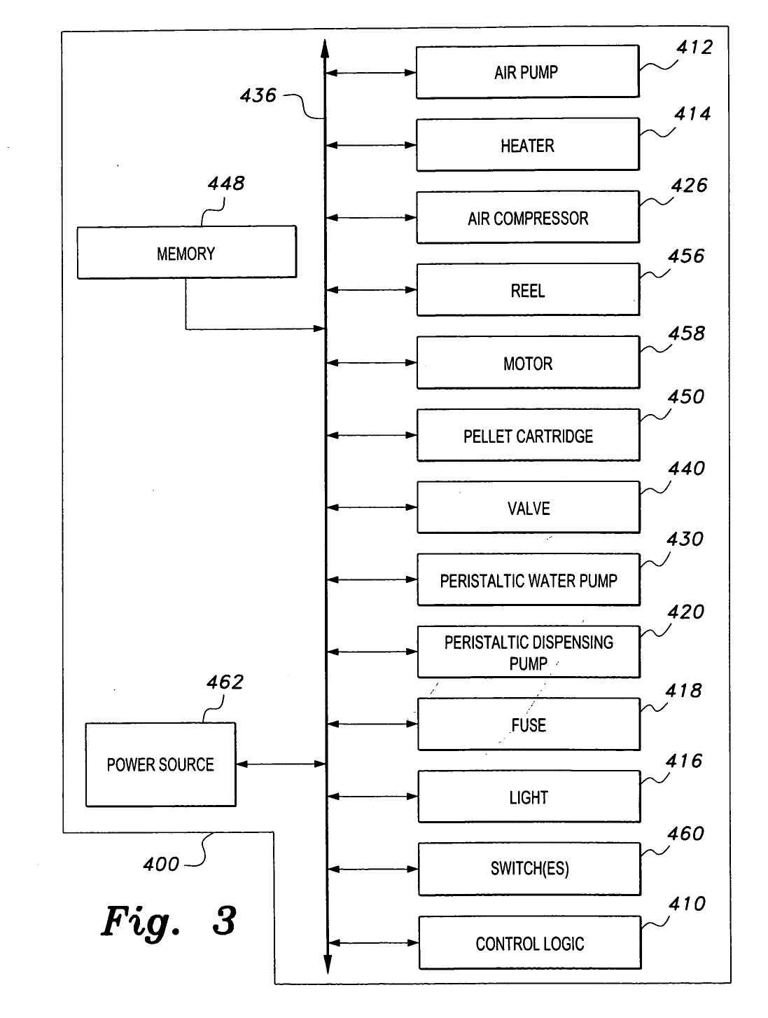

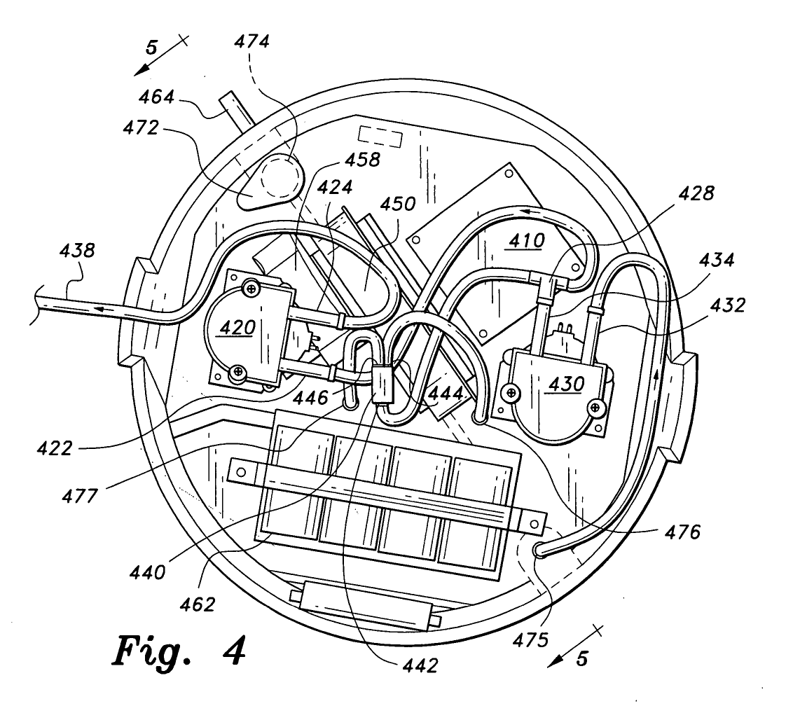

The system 100 is controlled and operated by controls disposed in the dispenser 400. Viewing FIG. 3 in conjunction with FIG. 4, the dispenser 400 includes switches 460 (e.g., an on-off switch, a manual override switch, a micro switch), an indicator light 416, a fuse 418, a peristaltic dispensing pump 420, a peristaltic water pump 430, a diverter valve 440, a pellet cartridge 450 which holds freeze-dried bacteria pellets 452, a small gear motor 458, a power source 462 and a control logic circuit 410. The control logic circuit 410 generates control instructions, and is in operable communication with the dispensing pump 420, the water pump 430, the diverter valve 440, and the gear motor 458, as well as a timer memory 448, via a communication bus 436. The dispenser 400 is preferably about 5½″ high and 11¼″ in diameter, but the dispenser 400 can have different dimension to function in different work locations.

The control logic circuit 410 may be realized as any number of devices and circuit configurations, but ideally includes a programmable central processing unit, such as a microprocessor, micro controller, programmable logic controller, or the like. The power source 462 used to run the system 100 can be 12V DC supplied by eight D-cell batteries or, alternatively, 115V AC using a step-down transformer rectified to provide (for example) 12V DC. The power source 462 used in the present invention is eight D-cell batteries in a battery holder that is strapped down to the dispenser 400 to prevent the batteries from accidentally falling out and pulling the terminals out of the dispenser 400. When the apparatus is run from an AC power source, the timer may include a lithium backup battery in case of power failure.

The dispenser 400 can optionally include an air pump 412, to aerate the dosing mixture and help with bacterial reproduction, a heater 414 controlled by a thermostat or thermistor that warms water pumped into the container 300 to a temperature ideal for bacteria growth and reproduction and an air compressor 426 that atomizes the dosing mixture.

The system 100 serves to generate the active bioremediation dosing mixture for a future day and contemporaneously dispense dosing mixture that was prepared the day before. The dosing mixture is prepared by adding a pellet 452 to water. The pellet 452 is manufactured from a mixture or consortium of multiple strains of vegetative, non-pathogenic, non-spore forming bacteria and, in certain applications, particular strains of spore forming bacteria that are freeze-dried and pulverized into a powder. The freeze-dried powdered bacteria is then combined with powdered nutrients and other lesser components and compressed into the pellet 452. The pellet 452 is preferably about ½″ in diameter and about 5/16″ wide, but can vary in size depending on how much of the pellet 452 is required to work in a specific application e.g. high volume applications or low volume applications. The dormant bioremediation bacteria contained within the pellet 452 becomes activated when the pellet 452 is added to water and allowed to proliferate. The powdered nutrients and other components contained in the pellet 452 provide nourishment to the growing bacteria, providing a high bacteria count in a single 24-hour period.

The dispenser 400 stores about thirty to thirty-one pellets 452 in the wheel cartridge 450. Alternatively, if the system 100 were designed The dispenser 400 stores about thirty to thirty-one pellets for high volume application then the wheel cartridge 450 could be designed to hold larger pellets 452, and smaller pellets 452 in low volume applications. Each pellet 452 is heat sealed in its own cavity by aluminum foil tape 454 or any suitable tape that blocks moisture from passing through to the pellet 452 to prevent pellet deterioration. The cartridge 450 sits on the outer periphery of the pan 300 just between the two chambers 320, 340 and is zigzag shaped. The shape of the wheel cartridge 450 is designed to allow one pellet 452 to sit over and fall into the only one of the chambers 320, 340 at one time. The cartridge 450 is preferably about 5″ in diameter and about 1″ thick, but can be made to have different dimensions based on the requirements of the system 100.

The chambers 320, 340 separate the dosing mixture made the previous day from water that will be used contemporaneously to prepare dosing mixture for the subsequent day. Chambers 320, 340 will alternate between functioning as a dispensing chamber that dispenses dosing material made in the previous cycle and as a generating chamber that receives water and the pellet 452 to make the dosing material. The pellet 452 is expelled from the cartridge 450 into the generating chamber by the movement of the gear motor 458. As the gear motor 458 turns, a take up reel 456 is propelled to turn, thereby winding the tape 454 off of the cartridge 450 and onto the reel 456 exposing one pellet 452. The gear motor 458 is controlled by switch 460, which is preferably a micro switch. The gear motor 458 runs for about twenty seconds per day and rotates at a speed of about 2 rpm. The cartridge 450 rotates about 11° to 11.8° once per day for each pull of the tape 454.

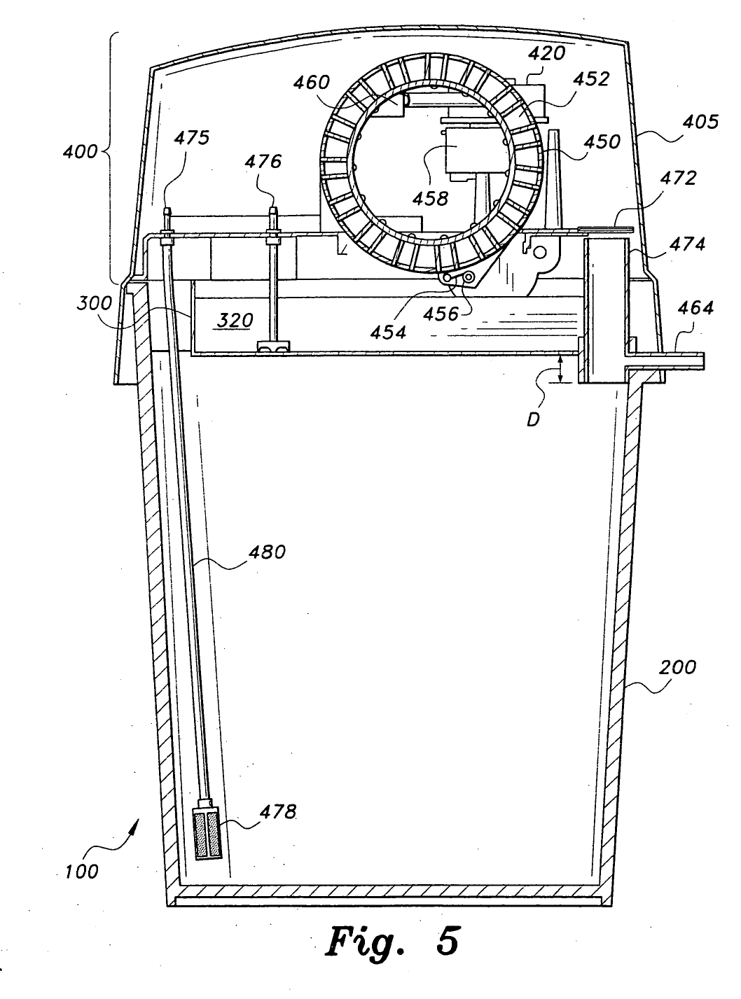

Referring now to FIG. 4 in conjunction with FIG. 5, as mentioned above, the system 100 might use water from any source to prepare the dosing mixture. Here, the pail 200 stores five gallons of water that is eventually pumped into the container 300 and dispensed out to the waste drain. To fill the pail 200 with water, a user lifts a hinged lid 405 on the dispenser 400 and a fill flap 472 sitting over the opening of an extension body 474. The extension body 474 forms a conduit from the top of the dispenser 400 into the cavity of the pail 200. A distance D must be maintained between the bottom of the pan 300 and the topmost level of the water stored in the pail 200.

The distance D is created and maintained by draining excess water out of the pail 200 through a filler body 464 before it can reach the pan 300. The filler body 464 is a tube-like structure that forms a drain port on the side of the pail 200. The filler body 464 is chemically bonded to the extension body 474. The user will know when to stop filling the pail 200 with water when excess water starts draining out of the filler body 464. If distance D is not maintained, and excess water is allowed to fill the pail 200 and reach the bottom of the pan 300, then the pan 300 would be dislodged from the top of the pail 200, causing it to float and interrupt the proper functioning of the system 100.

Once the system 100 is connected to a water source, such as the five gallons of water in the pail 200, the dispenser 400 can start to fill the container 300 with water in the designated generating chamber and dispense dosing material from the designated dispensing chamber.

The system 100 runs in 24-hour cycles. Each cycle begins once a day at a designated time, e.g., midnight. On the first day, water will be pumped into the designated generating chamber from the pail 200 but will not dispense dosing material from that chamber. Dosing material is normally dispensed after having completed at least one 24-hour cycle within which the pellet mixes with water in the chamber, becomes activated, feeds on the nutrients, and proliferates to form colonies of bioremediation bacteria in aqueous solution, thereby constituting the dosing material. After the first cycle the system 100 will both generate and dispense dosing material to the pipes P or other waste drain systems.

The control logic circuit 410 governs which chamber 320 or 340 will serve as the generating chamber and which serves as the dispensing chamber. The designated generating chamber receives water from the pail 200 via the water pump 430, while the designated dispensing chamber dispenses dosing material via the dispensing pump 420. Separate gear motors, typically 12V , drive both the dispensing pump 420 and the water pump 430 at about 125 rpm, pumping 1.18 ml/revolution.

The water pump 430 makes about 517 revolutions to pump the designated generating chamber with 610 ml of water. Therefore, at 125 rpm, pump 430 will run about 4.1-6 minutes each day. The dispensing pump 420, on the other hand, is on and off many times during the 24-hour period anywhere between about thirty seconds to one minute every two hours per day. The schedule for dispensing the dosing material into the drain is programmed into the control logic circuit 410. Ideally the pump 420 is programmed to discharge 51 ml of dosing material twelve times a day, once every two hours. The dispensing pump 420 turns off in about 20 seconds after about 52 revolutions. The system 100 is unable to dispense all of the dosing material out of the designated dispensing chamber, and as a result, a small amount of dosing material is left in the chambers for the next cycle.

When the control logic circuit 410 designates the generating chamber to be chamber 320, the water pump 430 draws 610 ml of water up from the pail 200 through a weighted filter or strainer 478, a tube 480, a filter head 475, and into an inlet port 432 of the water pump 430. The water is then pumped out an outlet port 434 of the water pump 430 through one exit end of a T-connector 428 and into a common port 442 of the diverter valve 440. The T connector 428 has two exit ends: one exit end connects to the common port 442 and a second exit end connects to an inlet port 422 of the dispensing pump 420. The T-connector 428 therefore connects the diverter valve 440 to both dispensing pump 420 and water pump 430.

Once water enters the diverter valve 440 through the common port 442, it can travel out one of two diverter exit ports 444, 446, either a normally open diverter exit port 444, or a normally closed diverter exit port 446, depending on which port 444, 446 is designated open by the control logic circuit 410. The diverter valve 440 is used to direct the routing of water into, or the drawing of dosing material out of, the chambers 320, 340. The diverter 440 is solenoid actuated and, as mentioned above, is controlled by the control logic circuit 410 to be active or deactivated.

If the diverter 440 is deactivated, then water exits the normally open port 444 and enters chamber 320 via a filter head 476. However, if diverter 440 is activated by the control logic circuit 410, the normally open port 444 is closed and water passes through the normally closed port 446, which is now open, into the chamber 340 via a filter head 477. The diverter 440 ensures that only one chamber 320, 340 receives water at one time.

In the present example, after water enters the common port 442, it leaves the exit port 444, travels through the filter head 476 and into the designated generating chamber, in this case chamber 320. As soon as chamber 320 is filled, the control logic 410 turns off the water pump 430, and directs the switch 460, e.g., the micro switch or an optic switch, to turn on the gear motor 458. The movement of the gear motor 458 propels both the reel 456 and the cartridge 450 to turn consequently releasing the pellet 452 to prepare the dosing material for the next 24-hour cycle.

As the chamber 320 is fermenting dosing material for the next cycle, chamber 340 is ready to dispense its previously prepared dosing material via the dispensing pump 420. The dispensing pump 420 is turned on to dispense dosing material after the water pump 430 is turned off, since only one pump can operate at any one time. The pump that is turned off serves as a check valve for the pump that is designated on. Accordingly, here the water pump 430 serves as a check valve for the operating dispensing pump 420.

To pump the dosing material out of the second chamber 340, the control logic circuit not only turns on the dispensing pump 420 and turns off the water pump 430, but also activates the diverter 440 so that the normally closed exit port 446 is open and the normally open exit port 444 is closed. The dosing material, therefore, is drawn out of the second chamber 340 through filter head 477, up the exit port 446, out the common port 442 and through the end of the T-connector 428 that is connected to the inlet port 422 of the dispensing pump 420. The dosing mixture is finally dispensed out to the pipes P or the drains via a dosing line 438 connected to an outlet port 424 of the dispensing pump 420.

The method of dispensing the dosing mixture via the dosing line 438 can be accomplished in numerous ways, such as by using peristaltic motion, air pressure or water pressure. A venturi device is used to dispense the bacteria by air or water pressure. If air pressure is used as the dispensation method, the dosing mixture is forced through an atomizer nozzle and dispensed in a fog. When water pressure is used, water is dispensed either in a spray form, a solid stream, or drops.

Generally the method for applying continuous bioremediation bacteria to the waste drain requires the dosing material to be prepared in the generating chamber while contemporaneously dosing the waste drain with active bioremediation bacteria mixture, prepared in the previous cycle, from the now designated dispensing chamber. After nearly all of the dosing material has been dispensed from the dispensing chamber into the pipes P the roles of the chambers switch wherein the previous cycle's generating chamber becomes the next cycle's dispensing chamber, and the previous cycle's dispensing chamber becomes the next cycle's generating chamber. The steps repeat after exhaustion of the dosing mixture from the dispensing chamber.

The system 100 is unique in its capacity to ferment/brew dosing material contemporaneously while dispensing dosing material made the previous day. This is unlike current bioremediation methods that require commercially available bacteria based products to be poured into drains. In this situation, problems exist, such as long waiting periods before use or requiring long activation times prior to the bacteria becoming activated. Here the bacteria degenerate before use or may only become active when flushed downstream.

As noted above, current bioremediation methods use an automated system called a bladder bag system (BBS). The BBS periodically injects a small amount of once prepared dosing material into drain lines, therefore continually restoring bacteria upstream. The BBS uses batches of dosing material that was prepared once prior to the start of a cycle; the cycle can be any period such as two to four weeks. A bacteria count test was conducted on the BBS, which showed the dosing material injected late in the cycle only contained a fraction on the amount of live active bacteria that existed early in the cycle.

An experiment was conducted to compare the growth and presence of bacteria between the bioremediation bladder bag system and the dual chamber bioremediation apparatus 100 of the present invention over the course of thirty days.

### Experiment

The experiment used three bladder bags to represent the BBS and three 1 L flasks to simulate the system 100. All six vessels were kept in a lab at room temperature of generally between 21° C. and 25° C. The bladder bags were kept in the lab in five gallon buckets, hydrated with five-gallons of water and capped. The caps were only removed once a day to extract samples and therefore reduce the likelihood of contamination.

The three 1 L flasks represent the container 300. The flasks were wrapped in parafilm to reduce the possibility of contamination and were only unwrapped once a day to take samples. Each of the three flasks was prepared once daily by measuring the exact amount of ingredients in the pellet and adding the ingredients and 610 mL of water to each flask. The experiments reused the same flasks throughout the entire test period to simulate the conditions of the system 100.

Samples were taken, at the same time each day from all six experiments and plated. After samples were taken from each flask, much of the dosing material in the flasks was discarded. However, the flasks retained a small amount of dosing material in the bottom of each flask to replicate the actual conditions of the system 100. During the normal operation of the system 100, neither chamber 320, 340 completely drains the dosing material at the end of each cycle.

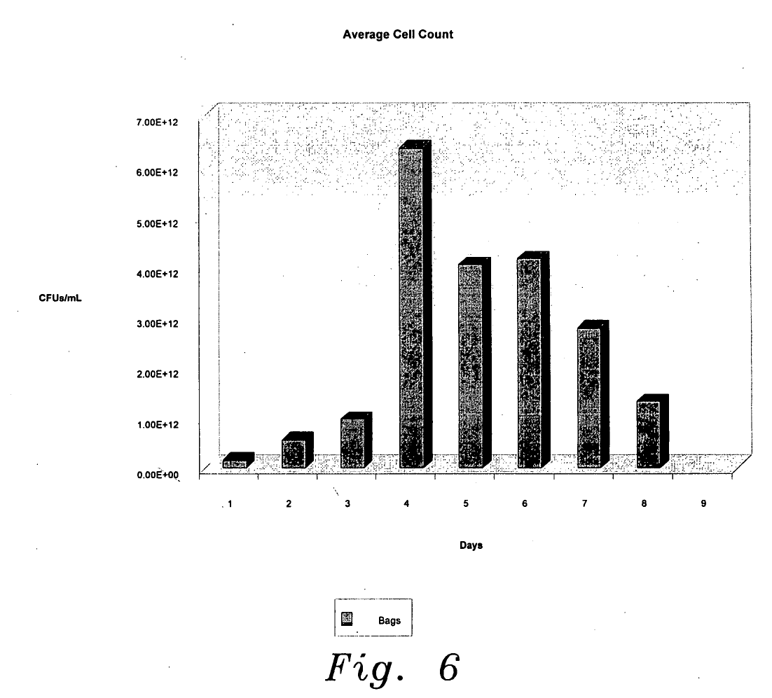

The six samples were all serially diluted in sterile DI water and 1% weight/volume peptone solution, dispensed on to Difco plate count agar using the Spiral Biotech Autoplate 4000 and then incubated at 30° C. for 18-24 hours. After incubation, the plates were manually counted to analyze for bacterial proliferation yielding data shown in Tables 1 and 2, and FIGS. 6-11.

### Results

TABLE 1

| Day | Bag #1 | Bag #2 | Bag #3 | Pellet #1 | Pellet #2 | Pellet #3 |
|---|---|---|---|---|---|---|
| 1 | 4.40 × 10¹¹ | 1.00 × 10¹⁰ | 1.40 × 10¹⁰ | 3.14 × 10¹⁰ | 1.42 × 10¹⁰ | 9.44 × 10¹⁰ |
| 2 | 8.00 × 10¹¹ | 8.00 × 10¹¹ | 8.00 × 10¹⁰ | 4.48 × 10¹¹ | 1.58 × 10¹¹ | 5.82 × 10¹¹ |
| 3 | 1.20 × 10¹² | 7.40 × 10¹¹ | 9.60 × 10¹¹ | 1.50 × 10¹¹ | 4.30 × 10¹¹ | 1.70 × 10¹¹ |
| 4 | NC | 4.54 × 10¹² | 8.16 × 10¹² | 1.68 × 10¹¹ | 3.22 × 10¹¹ | 9.20 × 10¹⁰ |
| 5 | NC | 6.48 × 10¹² | 1.60 × 10¹² | 3.20 × 10¹⁰ | 1.20 × 10¹² | 1.72 × 10¹¹ |
| 6 | 5.00 × 10¹² | 5.56 × 10¹² | 1.92 × 10¹² | 4.04 × 10¹¹ | 6.84 × 10¹¹ | 2.08 × 10¹¹ |
| 7 | 4.06 × 10¹² | 2.26 × 10¹² | 2.02 × 10¹² | 8.24 × 10¹¹ | 1.16 × 10¹² | 7.28 × 10¹¹ |
| 8 | 1.84 × 10¹² | 1.74 × 10¹² | 4.00 × 10¹¹ | 2.42 × 10¹¹ | 4.36 × 10¹¹ | 3.70 × 10¹¹ |
| 9 | 4.40 × 10¹¹ | NC | NC | NC | NC | NC |
| 10 | 4.00 × 10¹⁰ | 1.98 × 10¹¹ | 5.60 × 10¹⁰ | 4.20 × 10¹¹ | 4.28 × 10¹¹ | 1.70 × 10¹¹ |
| 11 | 1.28 × 10¹⁰ | 1.94 × 10¹⁰ | 6.60 × 10¹⁰ | 1.48 × 10¹¹ | 6.36 × 10¹¹ | 5.44 × 10¹¹ |
| 12 | 1.08 × 10¹⁰ | 5.40 × 10¹⁰ | 1.30 × 10¹⁰ | 1.94 × 10¹¹ | 3.46 × 10¹¹ | 4.98 × 10¹¹ |
| 13 | 1.44 × 10¹⁰ | 1.46 × 10¹⁰ | 1.70 × 10¹⁰ | 2.36 × 10¹¹ | 2.90 × 10¹¹ | 4.02 × 10¹¹ |
| 14 | 1.12 × 10¹⁰ | 4.26 × 10¹⁰ | 2.14 × 10¹⁰ | 2.60 × 10¹⁰ | 5.12 × 10¹¹ | 1.24 × 10¹² |
| 15 | NC | 1.60 × 10¹⁰ | 1.52 × 10¹⁰ | 2.36 × 10¹¹ | 3.14 × 10¹¹ | 3.92 × 10¹¹ |
| 16 | 1.78 × 10⁹ | 1.00 × 10⁹ | 6.60 × 10⁹ | 2.74 × 10¹¹ | 2.26 × 10¹¹ | 2.80 × 10¹¹ |
| 17 | 2.60 × 10⁸ | 5.40 × 10⁸ | 1.32 × 10⁹ | 2.22 × 10¹¹ | 3.34 × 10¹¹ | 3.46 × 10¹¹ |
| 18 | 2.70 × 10⁸ | 6.20 × 10⁸ | 9.40 × 10⁸ | 2.44 × 10¹¹ | 3.24 × 10¹¹ | 2.92 × 10¹¹ |
| 19 | 1.94 × 10⁸ | 5.20 × 10⁸ | 5.80 × 10⁸ | 2.84 × 10¹¹ | 3.62 × 10¹¹ | 3.54 × 10¹¹ |
| 20 | 2.16 × 10⁸ | 3.40 × 10⁸ | 4.20 × 10⁸ | 2.74 × 10¹¹ | 4.42 × 10¹¹ | 3.66 × 10¹¹ |
| 21 | 5.40 × 10⁷ | 4.40 × 10⁸ | 6.40 × 10⁸ | 2.28 × 10¹¹ | 2.36 × 10¹¹ | 4.40 × 10¹¹ |
| 22 | 6.80 × 10⁷ | 2.80 × 10⁸ | 6.20 × 10⁸ | 1.06 × 10¹² | 2.38 × 10¹¹ | 8.04 × 10¹¹ |
| 23 | 6.40 × 10⁷ | 2.80 × 10⁸ | 1.40 × 10⁸ | 6.80 × 10¹¹ | 2.78 × 10¹¹ | 8.12 × 10¹¹ |
| 24 | 7.20 × 10⁷ | 4.60 × 10⁸ | 8.20 × 10⁷ | 2.48 × 10¹¹ | 6.52 × 10¹¹ | 2.64 × 10¹¹ |
| 25 | 4.20 × 10⁷ | 3.60 × 10⁸ | 7.60 × 10⁷ | 2.38 × 10¹¹ | 5.32 × 10¹¹ | 2.56 × 10¹¹ |
| 26 | 5.40 × 10⁷ | 1.80 × 10⁸ | 5.80 × 10⁷ | 4.32 × 10¹¹ | 3.68 × 10¹¹ | 2.86 × 10¹¹ |
| 27 | 8.20 × 10⁷ | 9.40 × 107 | 6.60 × 10⁷ | 3.46 × 10¹¹ | 3.20 × 10¹¹ | 4.84 × 10¹¹ |
| 28 | 5.00 × 10⁷ | 6.80 × 107 | 2.60 × 10⁷ | 2.18 × 10¹¹ | 3.58 × 10¹¹ | 6.56 × 10¹¹ |
| 29 | 2.40 × 10⁷ | 2.00 × 10⁷ | 1.20 × 10⁷ | 2.22 × 10¹¹ | 7.24 × 10¹¹ | 5.72 × 10¹¹ |
| 30 | 1.80 × 10⁷ | 1.20 × 10⁷ | 8.00 × 10⁶ | 2.38 × 10¹¹ | 6.48 × 10¹¹ | 9.52 × 10¹¹ |

* “NC” signifies that no plate counts were taken that day *
* Measured in CFUs/mL

TABLE 2

| Day | Average for Bags | Average for Pellets |
|---|---|---|
| 1 | 1.55 × 10¹¹ | 4.67 × 10¹⁰ |
| 2 | 5.60 × 10¹¹ | 3.96 × 10¹¹ |
| 3 | 9.67 × 10¹¹ | 2.50 × 10¹¹ |
| 4 | 6.35 × 10¹² | 1.94 × 10¹¹ |
| 5 | 4.04 × 10¹² | 4.68 × 10¹¹ |
| 6 | 4.16 × 10¹² | 4.32 × 10¹¹ |
| 7 | 2.78 × 10¹² | 9.04 × 10¹¹ |
| 8 | 1.33 × 10¹² | 3.49 × 10¹¹ |
| 9 | NC | NC |
| 10 | 9.80 × 10¹⁰ | 3.39 × 10¹¹ |
| 11 | 3.27 × 10¹⁰ | 4.43 × 10¹¹ |
| 12 | 2.59 × 10¹⁰ | 3.46 × 10¹¹ |
| 13 | 1.53 × 10¹⁰ | 3.09 × 10¹¹ |
| 14 | 2.51 × 10¹⁰ | 5.93 × 10¹¹ |
| 15 | 1.56 × 10¹⁰ | 3.14 × 10¹¹ |
| 16 | 3.13 × 10⁹ | 2.60 × 10¹¹ |
| 17 | 7.07 × 10⁸ | 3.01 × 10¹¹ |
| 18 | 6.10 × 10⁸ | 2.87 × 10¹¹ |
| 19 | 4.31 × 10⁸ | 3.33 × 10¹¹ |
| 20 | 3.25 × 10⁸ | 3.61 × 10¹¹ |
| 21 | 3.78 × 10⁸ | 3.01 × 10¹¹ |
| 22 | 3.23 × 10⁸ | 7.01 × 10¹¹ |
| 23 | 1.61 × 10⁸ | 5.90 × 10¹¹ |
| 24 | 2.05 × 10⁸ | 3.88 × 10¹¹ |
| 25 | 1.59 × 10⁸ | 3.42 × 10¹¹ |
| 26 | 9.73 × 10⁷ | 3.62 × 10¹¹ |
| 27 | 8.07 × 10⁷ | 3.83 × 10¹¹ |
| 28 | 4.80 × 10⁷ | 4.11 × 10¹¹ |
| 29 | 1.87 × 10⁷ | 5.06 × 10¹¹ |
| 30 | 1.27 × 10⁷ | 6.13 × 10¹¹ |

* “NC” signifies that no plate counts were taken that day *
* Measured in CFUs/mL

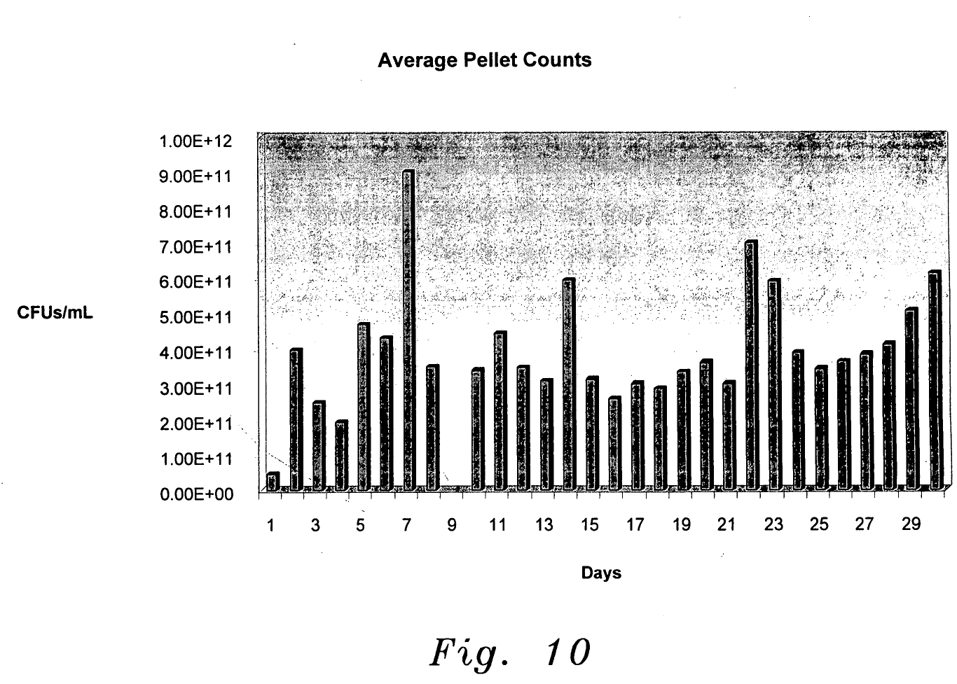

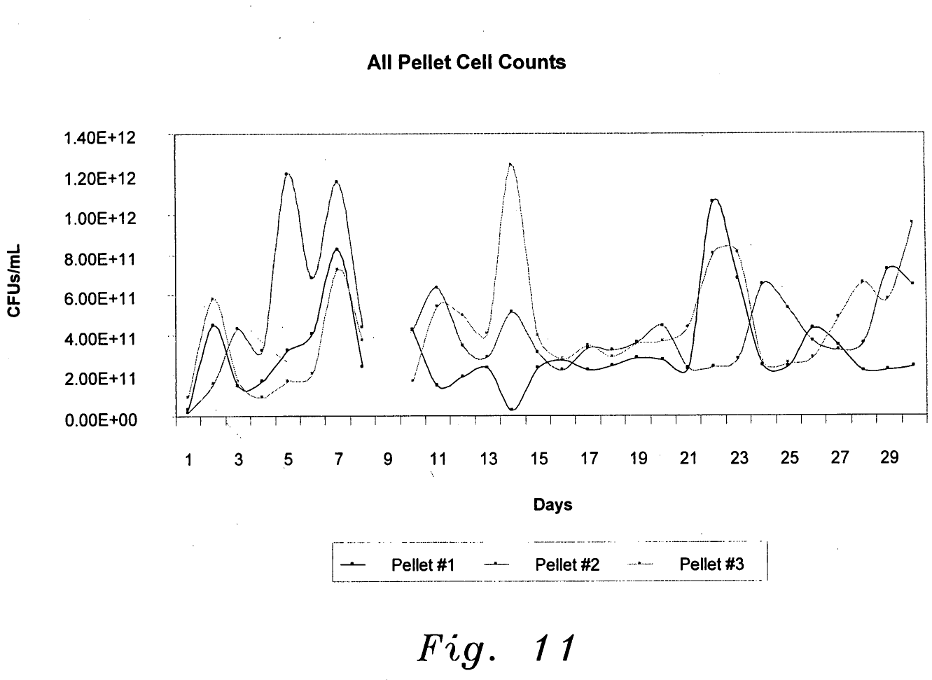

The cell counts for the bladder bags were initially high, reaching 10¹² CFUs/mL, but dropped to 10⁷ CFUs/mL toward the end of the test period. The largest decrease in growth occurred around mid-month. On the other hand, the cell counts for the simulated system 100 were lowest on the first day of the month, with the count for the remaining portion of the month being in the range of 10¹⁰ or 10¹², but being generally consistent at 10¹¹ CFUs/mL, (see FIG. 10). The overall picture of colonial growth for the pellets is a sinusoidal wave, (see FIG. 11).

The experiments were conducted in a controlled, generally constant environment with some fluctuations due to changes in air condition of the testing environment, location of experiment vessels, human error and inability to calculate the water left over in the flasks used to simulated the system 100.

The system 100, in comparison to the BBS, showed consistently higher cell counts throughout the course of one month due to the daily preparation and proliferation of dosing material. The BBS showed a decrease in cell count from 10¹² to 10⁷, being especially apparent during the last two weeks of the month, due to bacterial aging and nutrient depletion.

The value of the dual chamber bioremediation apparatus 100 is displayed in its effectiveness in maintaining high bacteria counts due to the brewing of dosing mixture one day prior to its dispensation.

Other advantages of the system 100 include ease in servicing, where a spent cartridge must simply be replaced and only the pan 300 needs to be cleaned. Also, unlike current bioremediation devices on the market, the system 100 deters users from diluting the dosing mixture to spread out its use over a cycle, since the dosing mixture must be made new each day and the amount of bioremediation bacteria dispensed is controlled in the form of a pellet 452. Furthermore, the system 100 is programmable, allowing the user to vary times and amounts of dosing mixture to be dispensed during a 24 hour cycle. In addition, unlike commercially made bioremediation mixtures that may have a long shelf life before use, the system 100 is prepared one day before use allowing the bioremediation bacteria to grow and reach generally peak proliferation prior to use.

It is to be understood that the present invention is not limited to the embodiments described above, but encompasses any and all embodiments within the scope of the following claims.

Higher bacteria counts may be achieved by adding water and the pellet 452 into the first chamber 320 every day, causing it to overflow into the second chamber 340 thereby designating the second chamber 340 as the dispensing chamber. For example, during day one, a day's supply of water and the pellet 452 would be dispensed into the first chamber 320. On day two, another day's supply of water and another pellet 452 would be added to the first chamber 320 causing it to overflow into the second chamber 340. At this point, both chambers 320, 340 would be full. Dispensing would then take place throughout day two. On day three, a third pellet 452 and a third day's supply of water would be added to the first chamber 320, again causing the first chamber 320 to overflow into the second chamber 340. At this point, the second chamber 340 is empty because its contents were dispensed on day two. This sequence would continue throughout the thirty-day cycle.

Modifications must be made to the system 100 to allow the system 100 to work with the overflow method just described. For instance, the wheel cartridge 450 would not be zigzag shaped and, as a result, the pellets 452 dispensed into the pan 300 would always fall into the first chamber 320. Also, a notch would have to be formed in a wall separating the chambers 320, 340 so the dosing mixture would spill over from the first chamber 320 to the second chamber 340. Additionally, the control logic circuit 410 would be programmed to always draw water from the water source into the first chamber 320 and always dispense the dosing mixture out of the second chamber 340.

Enhanced Replication and Bioremediation Apparatus and Method

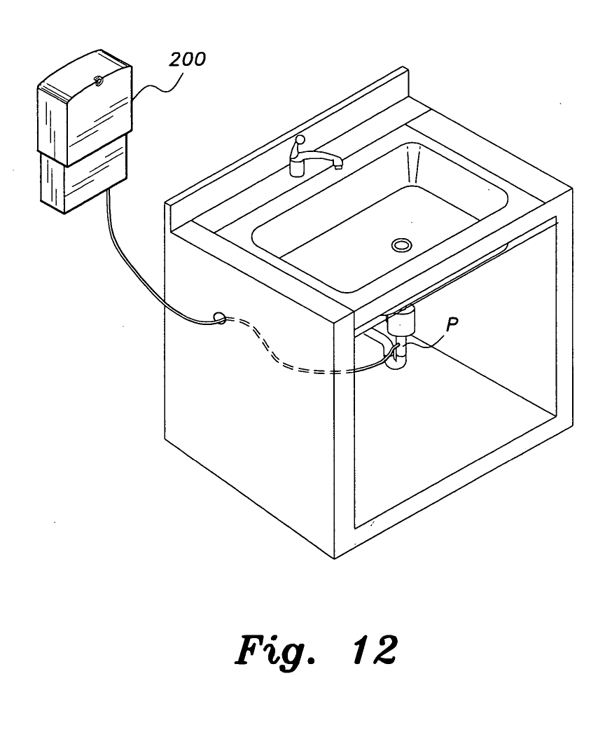

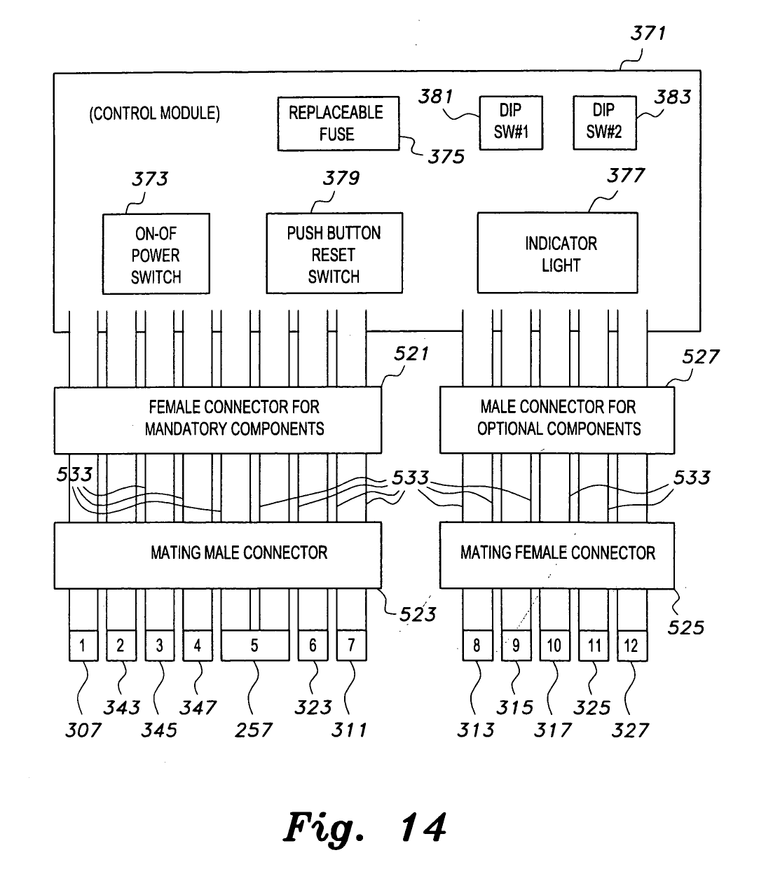

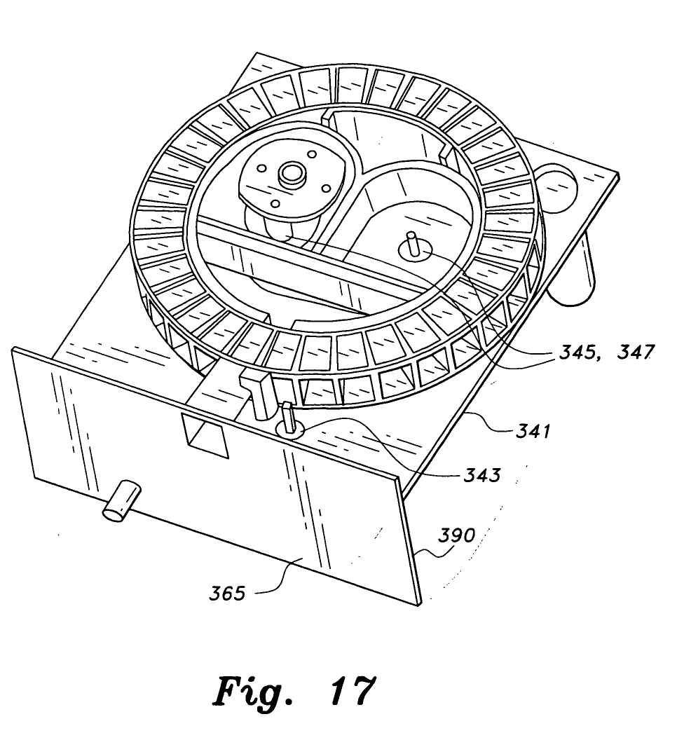

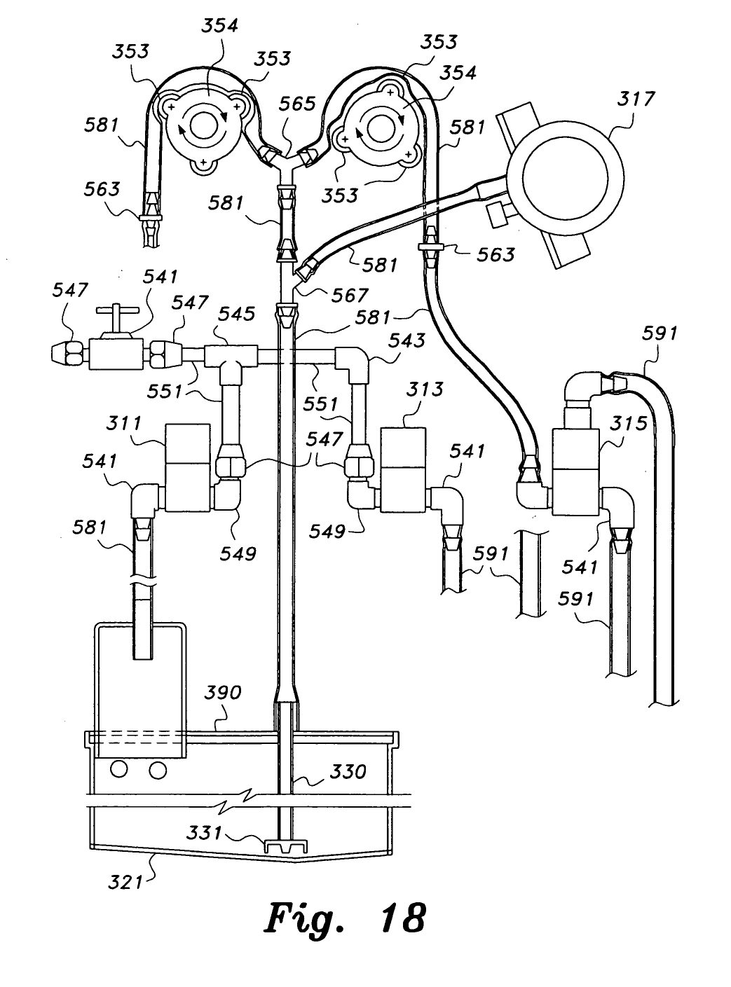

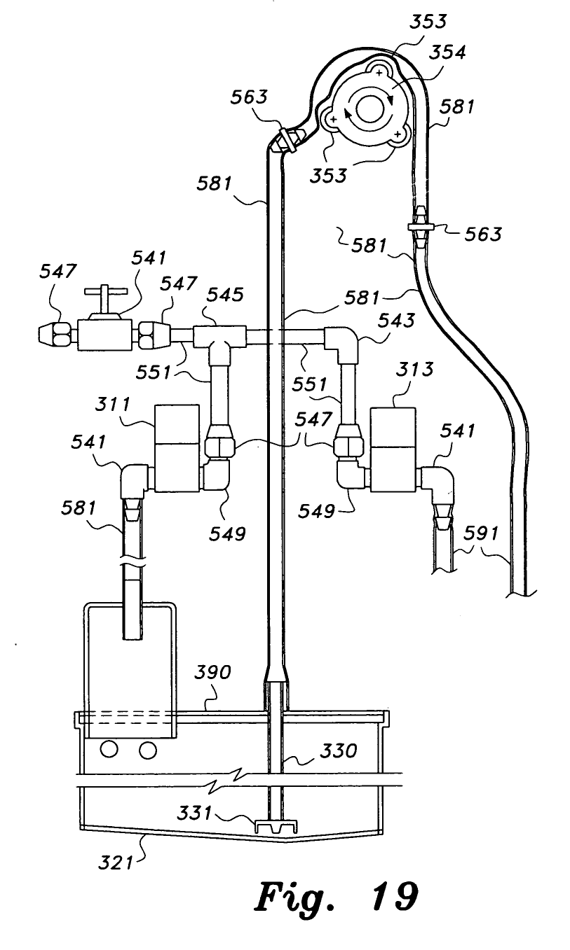

FIGS. 12 to 28 relate to the enhanced replication and bioremediation apparatus and method embodiments of the invention. FIGS. 19-28 relate to ten different configurations in which the system can operate to meet the user's needs.

The apparatus of the enhanced replication and bioremediation invention is virtually identical to the apparatus described for the dual chamber bioremediation invention except for the critical difference that the dual chambers of the dual chamber bioremediation invention are replaced by a single larger chamber sufficient to hold a forty-eight hour dosing supply of bioremediation dosing media. One critical element of the enhanced replication apparatus and method is the discovery that when only a twenty-four hour amount of dosing is withdrawn from the chamber that was initially filled with a forty-eight hour supply of dosing material,the rate of bacteria replication is substantially increased when a fresh pellet and a fresh 24 hour supply of water is added to fill the chamber, replacing the withdrawn dosing media. The result is a very high bacterium remediation count in the measured dosing media passed into the waste drain.

Ten Various Configurations to Meet Different Needs

The dispenser can be configured in any of 10 ways to best meet the specific need of the end-user as summarized below and explained in greater detail as follows:

The elements of the configuration of FIG. 19 include a transformer, a water line, pump 351, and before and after dispense flush.

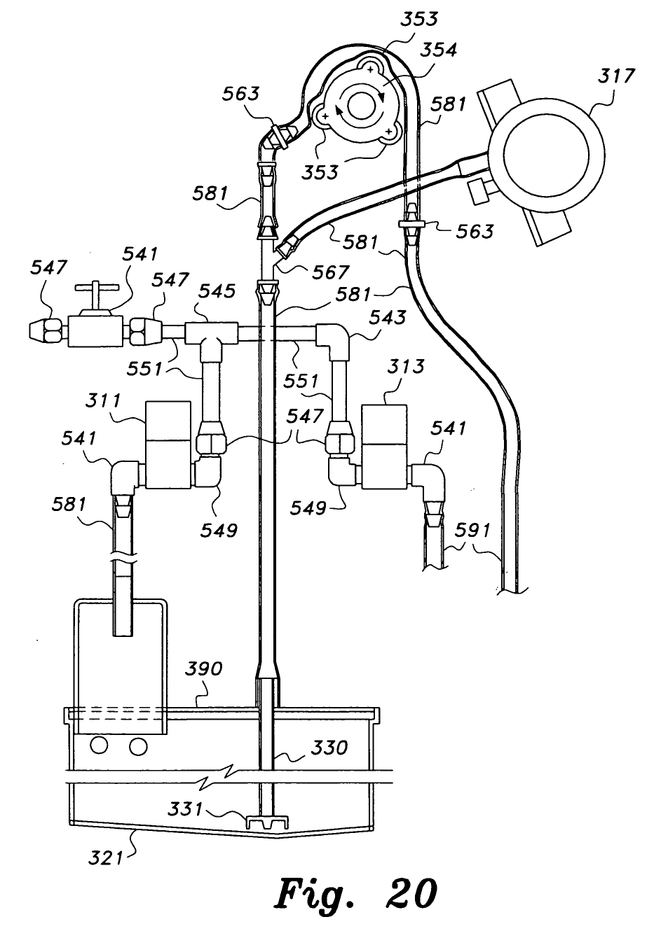

The elements of the configuration of FIG. 20 include a transformer, a water line, pump 351, before and after dispense flush and addition of oxygen via air pump.

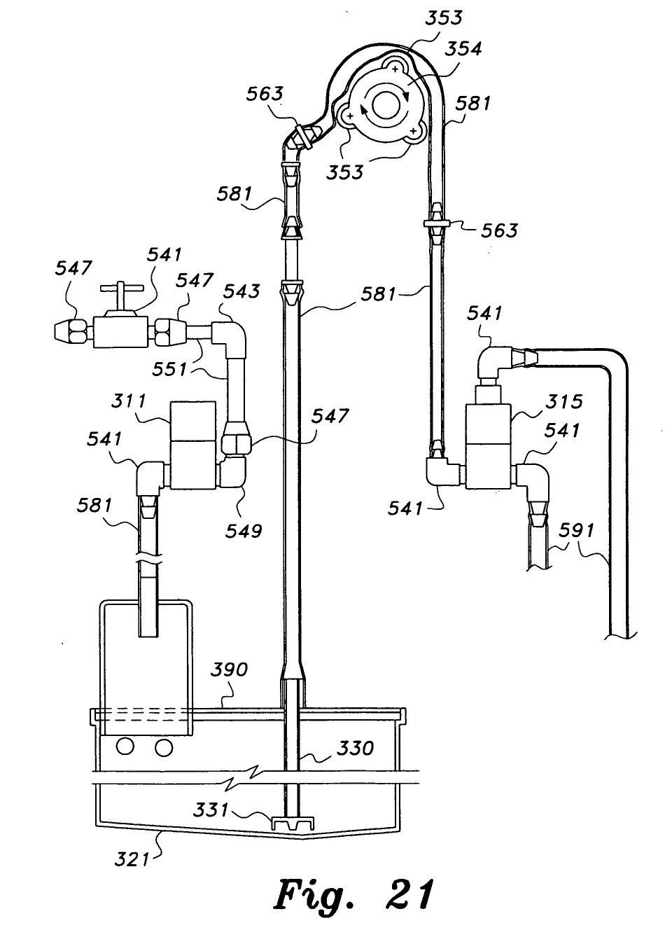

The elements of the configuration of FIG. 21 include a transformer, a water line, pump 351, and treatment of two drains.

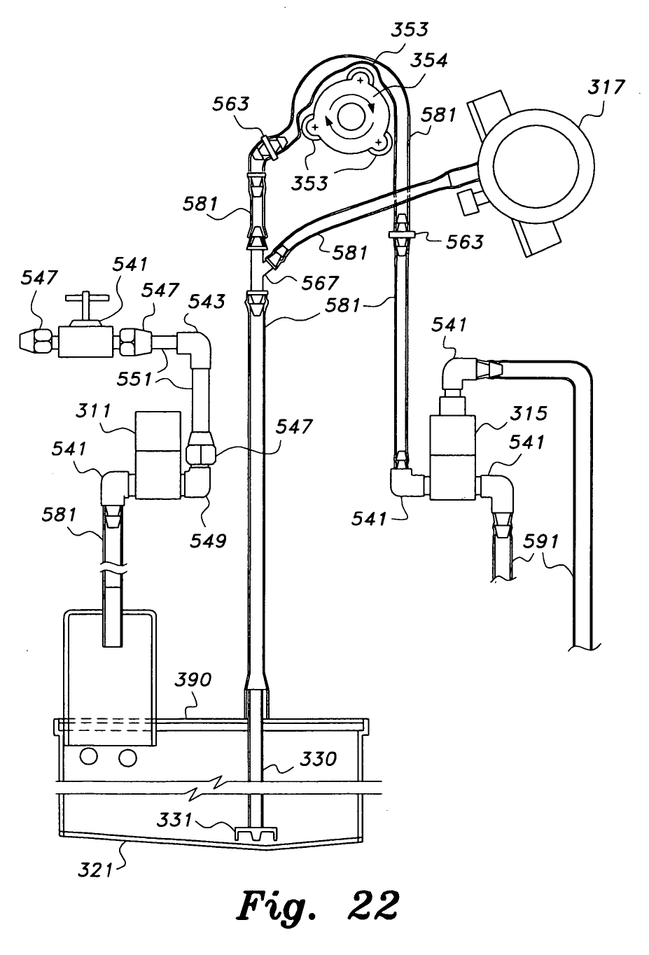

The elements of the configuration of FIG. 22 include a transformer, a water line, pump 351, treat two drains and add oxygen by air pump.

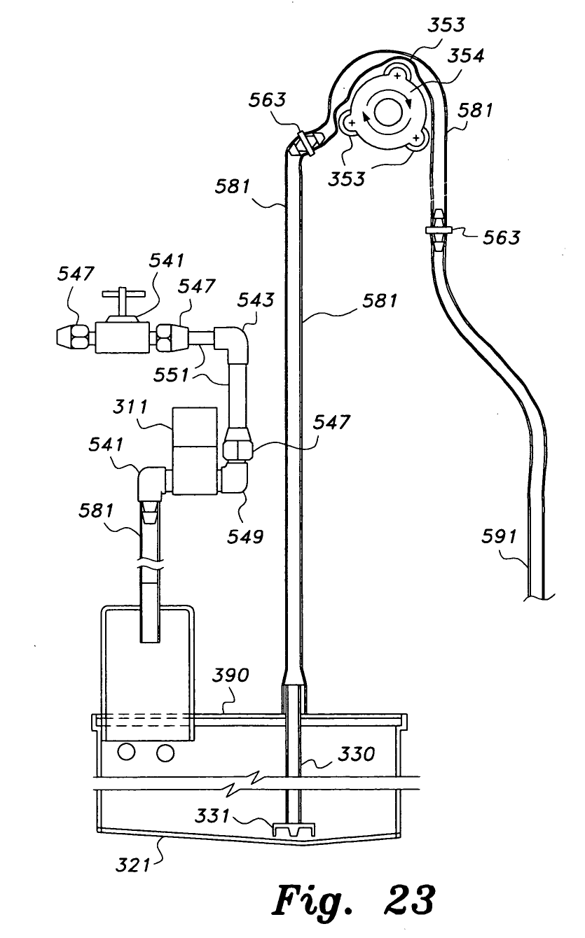

The elements of the configuration of FIG. 23 include a battery, a water line, and a pump 351.

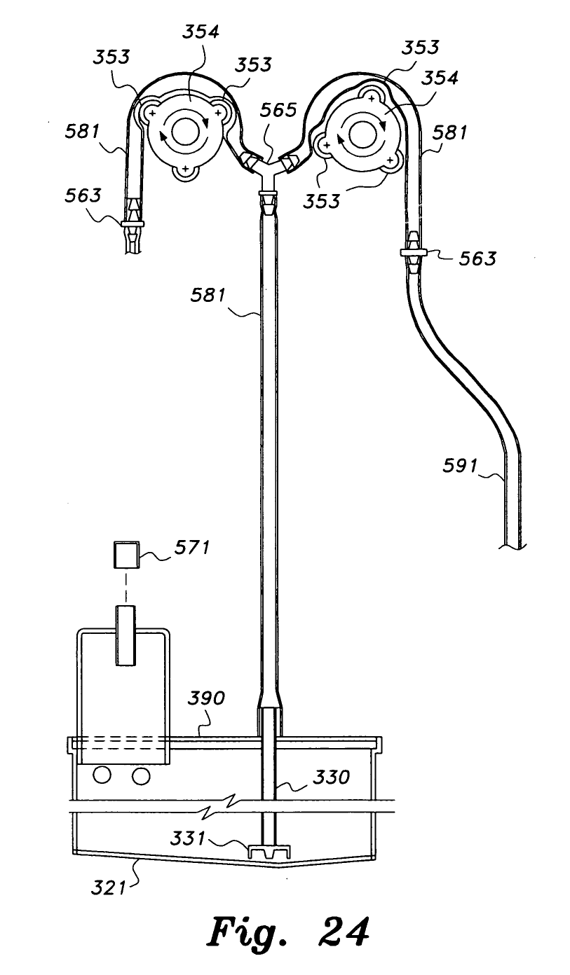

The elements of the configuration of FIG. 24 include a battery, a water reservoir, two pumps 351 and 349.

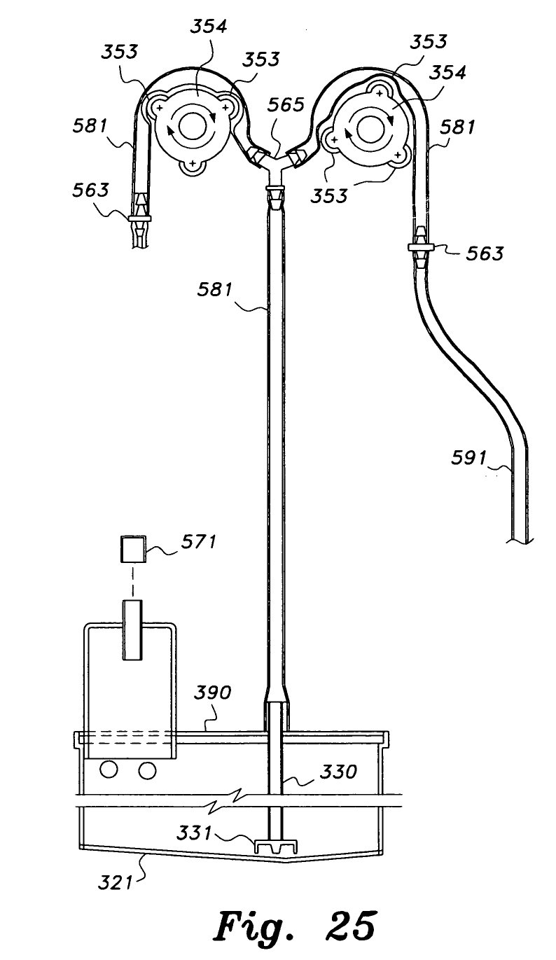

The elements of the configuration of FIG. 25 include a transformer, a water reservoir, and two pumps 351 and 349.

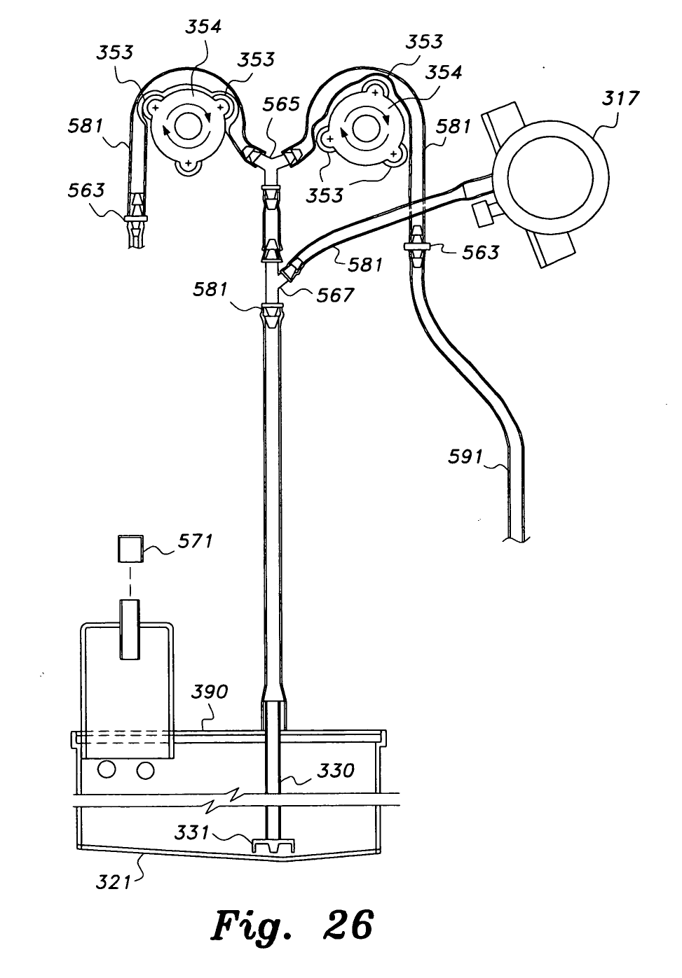

The elements of the configuration of FIG. 26 include a transformer, a water reservoir, two pumps 351 and 349 and an air pump for oxygen addition.

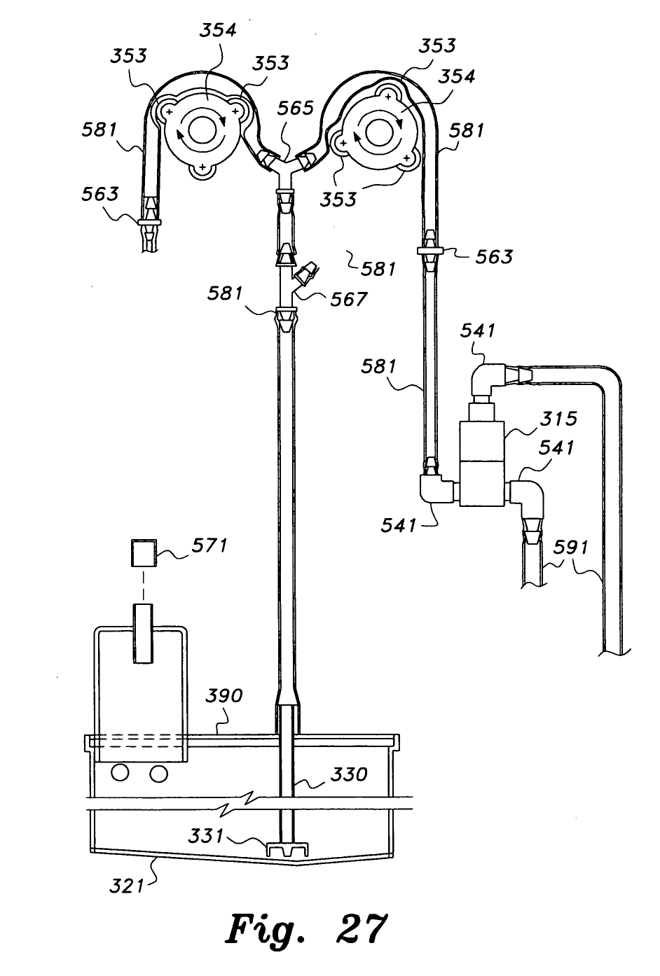

The elements of the configuration of FIG. 27 include a transformer, a water reservoir, two pumps 351 and 349 and treatment of two drains.

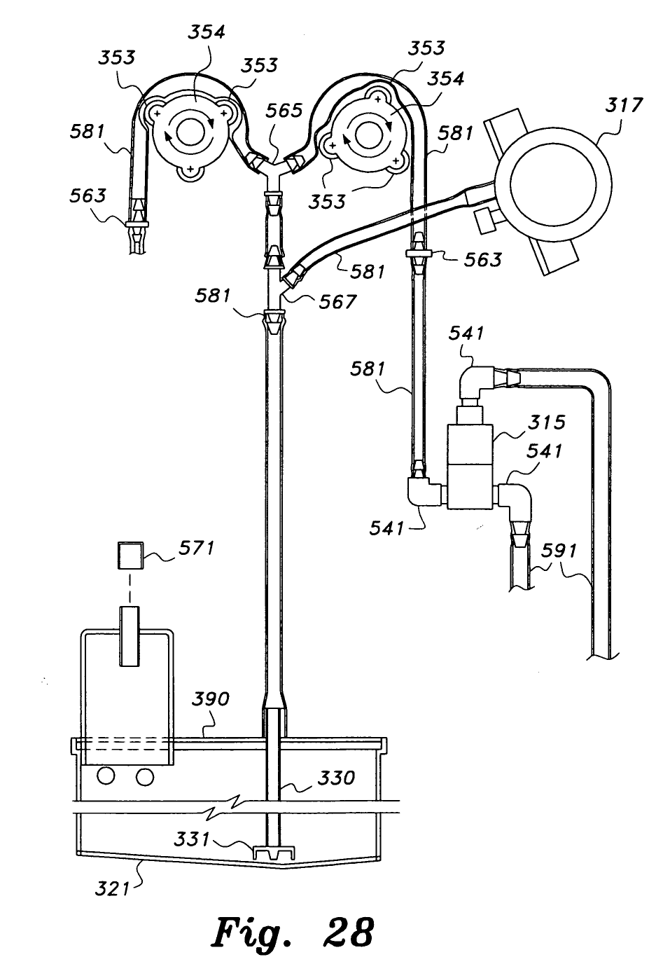

The elements of the configuration of FIG. 28 include a transformer, a water reservoir, two pumps 351 and 349, treatment of two drains and addition of oxygen by an air pump.

The dispenser 200 can be powered by battery pack 307 or by a wired power source such as an 110 vac, 60-hertz wall outlet through the use of a step-down transformer with the dispenser operating on a lower voltage, such as 24 vac. It can be configured to enable it to operate in locations outside the U.S. when the available power source may be 220V and/or the current 50-hertz, for example, by selecting the appropriate step-down transformer. It should be noted that certain electrical components are common to both battery powered and transformer-powered configurations, but others are not.

Except when a component is specific to one or the other, no distinction is made since such information is not relevant to the explanations and illustrations. The dispenser can be configured to use a reservoir (such as a 5-gallon bucket) or to use a pressurized water line for its water source.

When the power source is a wall outlet, it can also:

a. be configured with a small heater to keep the dosing material at the ideal temperature for the bioremediation bacteria to multiply. This is especially useful in colder climates where the dispenser might be located in a cold area that would otherwise result in slower operations;

b. be configured with an air pump to add oxygen to the dosing material, thereby accelerating replication activity;

c. be configured with a diverter valve that enables the dispenser to alternately dispense dosing material to two separate targeted

d. be configured with a pre-dispense and/or post-dispense capability, when the water source is a pressurized line, which allows tap water to be directed to the targeted destination (typically a drain line) either before the dispensing event to flush out any waste chemicals in the line that would be harmful to the soon to be dispensed bacteria or after the dosing event to move the dosing material further down line. This is sometimes beneficial if, as examples, the drain line traverses a long distance before reaching the grease trap. It is desirable that the leading edge of the dosing material reach the grease trap, thereby leaving a trail of bioremediation bacteria the entire length of the drain line that is being treated or when the drain line being treated is seldom used and would therefore be relatively dry, thus the need to inject water to help carry the dosing material further downstream. Either or both of the flushes (pre-dispense or post-dispense) can be disabled by turning off either or both of the dipswitches located on the control module.

As noted earlier, current bioremediation methods use an automated system called a bladder bag system. This system periodically injects a small amount of once prepared dosing material into drain lines, thereby continually restoring bioremediation bacteria at the treatment site. The bladder bag system uses batches of dosing material prepared prior to the start of a cycle; the cycle can be any period such as two or four weeks. A bioremediation bacteria count test was conducted on the bladder bag system, which showed a given volume of the dosing material injected late in the cycle contained only a fraction of the amount of live bioremediation bacteria that existed in the same volume of dosing material that was dispensed early in the cycle.

Operational Sequences and Interaction of Key Components Under the Ten Different Configurations

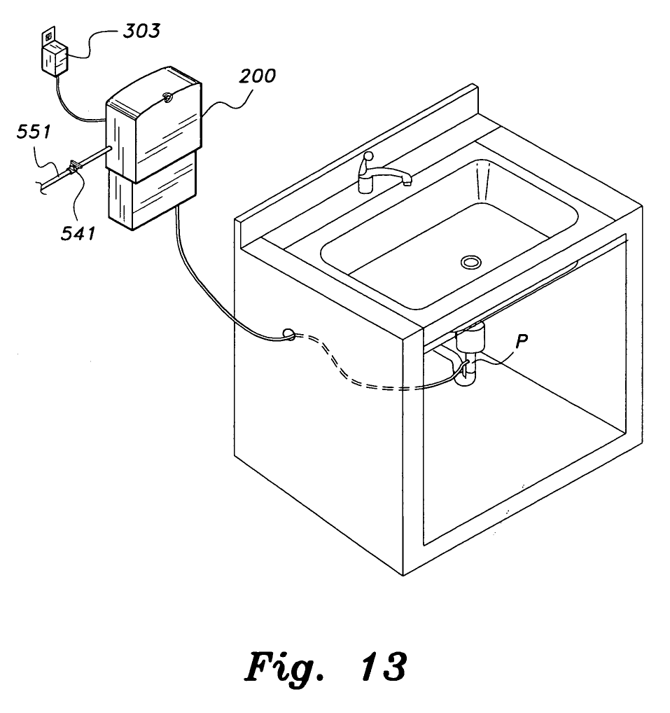

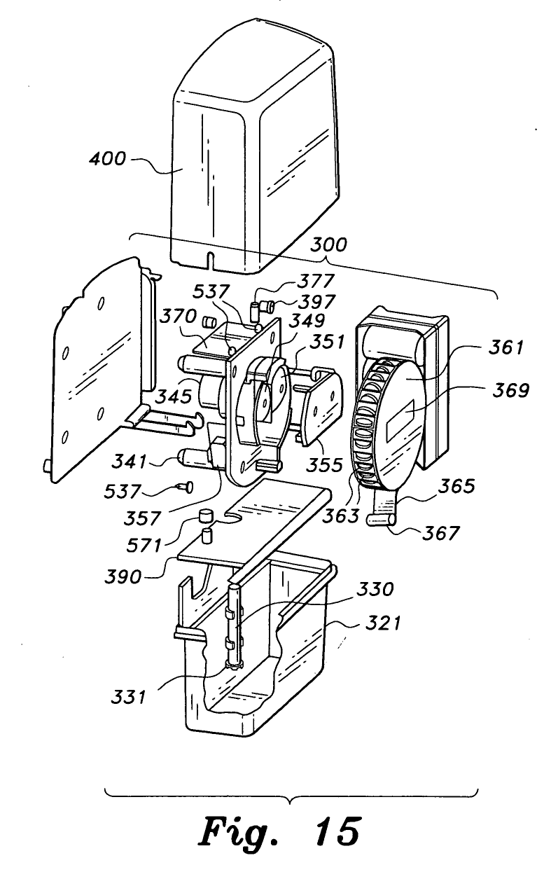

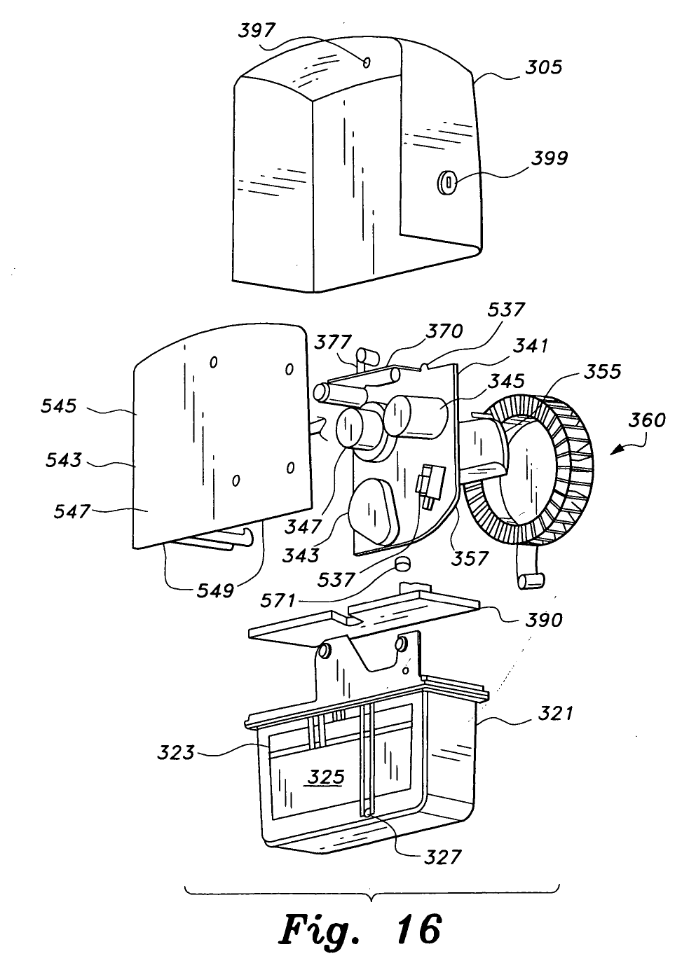

The operational matters are applicable to both battery pack 307 and transformer 303-powered dispenser 200 (Fig.13). Referring to FIG. 15 an FIG. 16, replication chamber 321 is filled when control module 370 causes gear motor 345 to be energized which in turn drives peristaltic pump 349 causing it to draw water from reservoir 150 (not shown), through said peristaltic pump 349. Since peristaltic pump 351 is stationary and therefore acts as a check valve, it dispenses the water into replication chamber 321. Liquid level detector 323 (not shown) senses when replication chamber 321 is full and sends a signal to control module 370 (not shown).

The following operational matters are applicable to both battery pack 307 and transformer 303 powered dispenser 200. An alternate means of filling replication chamber 321 is when a connection is made to a pressurized water line, referring to FIG. 20. Control module 370 causes 2-way NC valve 311 to be opened allowing water to flow through 2-way NC valve 311 and into replication chamber 321. Liquid level detector 323 senses when replication chamber 321 is full and sends a signal to control module 370, which de-energizes 2-way valve 311, allowing it to close. When this method is used, peristaltic pump 349 is omitted from dispenser 200 with the inlet of peristaltic pump 323 connected by tube 581 to fill/dispense tube 330.

The following operational matters are applicable to transformer 303 powered dispenser 200 only. Referring to FIG. 22, air pump 317 is optional, but used when there is a need to boost the bio-remediation bacteria counts by adding oxygen to the dosing material. Air pump 317 (not shown) is energized by control module 370 only when no other components with a high current draw, e.g. gear motors (FIG. 16) 343, 345, 347, valves 311, 313, 315 (FIG. 19) and heater 325, are energized. When air pump 317 is not used, branch wye fitting 567 is omitted and tube 581 leading from fill/dispense tube 330 (FIG. 19) to the next fitting either connector 563 at the inlet of peristaltic pump 351 or barbed wye connector 565, depending on the configuration is continuous and one piece.

The operational matter is applicable to both battery pack 307 and transformer 303 powered dispenser 200. To dispense dosing material from replication chamber 321, control module 370 energizes gear motor 347 causing it to drive peristaltic pump 351 and since peristaltic pump 349 is stationary and therefore acts as a check valve, draws dosing material from replication chamber 321 and dispenses it into the drain line.

The operational matters are applicable only to transformer 303 powered dispenser 200 that is connected to a pressurized water line (FIG. 21). Diverter valve 315 is optional installed only when there is a need to alternatively treat two different lines. This option is sometimes needed when two separate drain lines, both needing treatment, are near to each other and join into a common line before reaching the grease trap. When this option is installed, the output of peristaltic pump 351 is attached to the inlet port of diverter valve 315 and the outputs of diverter valve 315 are alternated by control module 370 from one dosing event to the next.

The operational matter are applicable only to transformer 303 powered dispenser 200 that is connected to a pressurized water line (FIG. 20). Pre-dispense and post-dispense flushing of the drain line is an option that requires 2-way NC valve 313 (FIG. 20). Either or both of the flushing events can be disabled by placing the controlling dip switch or switches 381, 383, located on control module 370, into the “off” position. Just prior to the dosing event, control module 370 opens 2-way NC valve 313 for a predetermined time (typically 10 seconds), allowing water from the pressurized line to flow directly to the drain line. Immediately after the dosing event, control module 370 energizes 2-way NC valve 313 for a pre-determined time (typically 5 seconds), allowing water from the pressurized line to flow directly to the drain line.

Referring to FIG. 24, a preferred configuration of the apparatus of the invention is illustrated. The apparatus employs a water reservoir of about five-gallon capacity and is battery powered. Two peristaltic pumps (354) are connected to the feed tube 581 attached to the pumps by wye diverter 565.

The present invention is directed to an apparatus and method for applying bioremediation bacteria repeatedly and frequently to a targeted destination, typically a waste drain or drainage system to break down grease and fatty residue, sugar, starch, cellulose and other waste products generated by processing food products.

Pellet Replication System

Control module 370 energizes gear motor 343 causing said motor to rotate take-up reel 367 which, being permanently bonded to the loose end of moisture barrier film 365, pulls the film around a fulcrum pin and onto take-up reel 367. This action causes pellet wheel 361 to rotate, uncovering pellets 363 one at a time. The program within control module 370 is such that on day one of the 31-day service cycle, gear motor 343 is de-energized after two pellets 363 have been allowed to drop into replication chamber 321 below. Every day thereafter throughout the remainder of the service cycle (typically 31 days), only one pellet 363 is allowed to drop into replication chamber 321. Following the dropping of pellet(s) 363, replication chamber 321 is filled by one of two ways: a. 2-way fill valve 311 is opened and tap water flows into replication chamber 321 or; b. gear motor 345 is energized by control module 370 causing peristaltic pump 345 to draw water from reservoir 150 and into replication chamber 321.

Regardless of which method is used, when liquid level detector 323 senses that replication chamber 321 is full, it sends a signal to control module 370 that in turn de-energizes the controlling device (2-way valve 311 or gear motor 345), stopping the water flow. Later, (typically two (2) hours) after replication chamber 321 has been filled, and continuing on a regular basis (typically every 2 hours), control module 370 will cause a dosing event to take place. The dosing event may be as simple as gear motor 347 being energized thereby driving peristaltic pump 351, causing dosing material to be dispensed directly into the targeted destination (typically a drain line) or (when transformer 303 powered and the 2-drain option is installed as part of the dispensing system, through the 2-drain diverter valve to the targeted destination. When the flushing option is installed and dip switch 381 in the “on” position, control module 370 causes 2-way valve 313 to be energized which opens the valve allowing pressurized tap water to flow directly into the targeted destination (typically a drain line). After a pre-determined time (programmed into control module 370), said control module 370 causes 2-way valve 313 to be de-energized, allowing it to return to the closed position, stopping the water flow. Control module 370 then causes the gear motor 347 that drives peristaltic pump 351 to be energized, thereby driving said peristaltic pump 351 and causing dosing material to be drawn from replication chamber 321 through foot filter 331, filler tube 330 and plastic tubing 591 to the targeted destination, typically a drain line. When the 2-drain option is installed, tube 581 from the output of peristaltic pump 351 is connected to diverter valve 315 which is then energized every second dosing event, thereby directing the dosing material alternately between two different targeted destinations, (typically two different drain lines). Control module 370 controls the amount of dosing material dispensed by counting the number of revolutions made by peristaltic pump 351 and when the programmed number is attained, gear motor 347 that drives peristaltic pump 351 is de-energized.

### Experiment

An experiment was conducted to compare growth and presence of bioremediation bacteria between the bladder bag system and the enhanced replication bioremediation apparatus and method of the present invention over the course of thirty days.

The experiment used three bladder bags to represent the bladder bag system and three flasks with a capacity of >1.2 L to simulate system 100. All six vessels were kept in a lab at room temperature. The bladder bags were kept in the lab in five gallon buckets, hydrated with five-gallons of water and capped. The caps were removed only once a day to extract samples and therefore reduce the likelihood. The three flasks that simulated replication chamber 321 were wrapped in Para film to reduce the possibility of contamination and were unwrapped only once a day to take samples. On day 1, 1.2 L of tap water was placed in each flask along with the exact amount of ingredients in two pellets. Approximately twenty-four hours later and every 24 hours thereafter throughout the 30-day test cycle, samples were extracted from each flask, then enough of the dosing material was discarded to leave 610 mL in each flask, followed by the exact amount of the addition of enough tap water to bring the volume of material in each flask back to 1.2 L. The experiments reused the same flasks throughout the entire test period to simulate the conditions.

Samples were taken at the same time each day from all six experiments and plated. The six samples were all serially diluted in sterile DI water and 1% weight/volume peptone solution, dispensed on to Difco plate count agar using the Spiral Biotech Autoplate 4000 and then incubated at 30° C. for 18-24 hours. After incubation, the plates were manually counted to analyze for bacterial proliferation yielding data. The results of the experiment are shown on the page following.

| DAY | BLADDER BAG | MGDS simulation |
|---|---|---|
| 1 | Not enough growth to count | 1.00E+07 |
| 2 | 5.60E+11 | 1.000E+10 |
| 3 | 9.67E+11 | 1.00E+11 |
| 4 | 6.35E+12 | 1.00E+12 |
| 5 | 4.04E+12 | 5.64E+12 |
| 6 | 4.16E+12 | 5.16E+12 |
| 7 | 2.78E+12 | 4.36E+12 |
| 8 | 1.33E+12 | 4.88E+12 |
| 9 | 1.24E+11 | 5.52E+12 |
| 10 | 9.80E+10 | 1.00E+13 |
| 11 | 3.27E+10 | 1.00E+13 |
| 12 | 2.59E+10 | 2.08E+12 |
| 13 | 1.53E+10 | 1.94E+12 |
| 14 | 2.51E+10 | 5.93E+11 |
| 15 | 1.56E+10 | 3.14E+11 |
| 16 | 3.13E+09 | 2.64E+12 |
| 17 | 7.07E+08 | 2.84E+12 |
| 18 | 6.10E+08 | 2.20E+12 |
| 19 | 4.31E+08 | 1.00E+13 |
| 20 | 3.25E+08 | 2.26E+12 |
| 21 | 3.78E+08 | 1.22E+12 |
| 22 | 3.23E+08 | 4.56E+12 |
| 23 | 1.61E+08 | 4.52E+12 |
| 24 | 2.05E+08 | 3.16E+12 |
| 25 | 1.59E+08 | 3.42E+11 |
| 26 | 9.73E+07 | 3.62E+11 |
| 27 | 8.07E+07 | 1.00E+13 |
| 28 | 4.80E+07 | 2.66E+12 |
| 29 | 1.87E+07 | 3.32E+12 |
| 30 | 1.27E+07 | 1.18E+12 |

The cell counts for the bladder bags were initially high, reaching 1012 CFUs/mL, but dropped to 107 CFUs/mL toward the end of the test. The largest decrease in growth occurred around mid month. On the other hand, the cell counts for the simulated system 100 with enhanced replication were lowest on the first day of the month, with the count for the remaining portion of the month being in the range of 1010 or 1013, but being generally consistent at 1012 CFUs/mL. The overall picture of colonial growth for the pellets is a sinusoidal wave. The experiments were conducted in a controlled, generally constant environment with some fluctuations due to changes in air condition of the testing environment, location of experiment vessels, human error and inability to calculate the water left over in the flasks. The system 100 with enhanced replication, in comparison to the bladder bag system, showed consistently higher cell counts throughout the course of one month due to the daily preparation and proliferation of dosing material. The bladder bag system showed a decrease in cell count from 1012 to 107, being especially apparent during the last two weeks of the month, due to bacterial aging and nutrient depletion.

The bioremediation bacteria used in the system 100 is manufactured from a consortium of multiple strains of vegetative, non-pathogenic, non-spore forming bacteria and, in certain applications, particular strains of spore forming bacteria that are freeze-dried and pulverized into a powder. The freeze-dried powdered bacteria is then combined with powdered nutrients and other lesser components and compressed into pellet form. The pellet is preferably about ½″ in diameter and about 5/16″ wide, but can vary in size depending on how much of the pelletized material is required to work in a specific application e.g. high volume designed for high volume applications then the wheel cartridge could be designed to hold larger pellets, and smaller pellets in low volume applications. The cartridge is preferably about 5″ in diameter and about 1″ thick, but can be made to have different dimensions based on the requirements of the system 100.

Although there are spore form bacteria strains that are very effective at bioremediation, they are used only to supplement the non-spore strains used in the system 100, simply because of their non-predictability as to when they can be enticed to come out of dormancy and become effective bioremediators. Commercially available bacteria based products can be poured into drains. Problems exist in such a scenario, including uncertainty that a spore form bacteria can be enticed to come out of dormancy and even if they do, an unpredictability factor as to how long the waiting periods before use or requiring long activation times prior to the bacteria becoming active. The risk is that the bacteria degenerate before use or may only become active after they have been carried far downstream.

A volume 8 ½″w×11″h×4 ½″d incorporates a chamber large enough to contain a 2-day supply of dosing material. A pellet wheel dispenses pellets containing a freeze-dried medium of multiple strains of bacteria and nutrients into the chamber. Water is drawn into the chamber and the media is allowed to germinate and proliferate colonies of bacteria for up to twenty-four hours to form a dosing material.

Simultaneously, dosing material is dispensed from the chamber into a drain line of a food preparation establishment at timed intervals to break up fats and grease in the drain, thereby preventing blockages. Additional pellet containing bacteria, nutrient and ingredients along with enough water to bring the chamber to full, said remaining dosing material acts as seed bacteria with the result that the replication process is enhanced, resulting in a much higher concentration of bio-remediation bacteria in the dosing material when it is dispensed from the replication chamber and into the targeted destination than it would be without this enhancement. The method involves enriching a current batch of bioremediation bacteria while simultaneously dispensing from the same chamber.

The invention is not limited to the embodiments described above, but encompasses any and all embodiments within the scope of the following claims.

1. A dual chamber bioremediation apparatus for applying bioremediation bacteria to a waste drain, comprising:
a container having a dividing wall defining a first chamber and a second chamber;
a pellet delivery mechanism positioned to deliver a pellet containing a freeze-dried bioremediation bacteria medium into the container;
a water delivery mechanism connected to the container for delivering a metered quantity of water from a water source to the container;
a dispenser mechanism attached to the container and adapted for connection to the waste drain; and
a control logic circuit programmed to alternately switch the pellet delivery mechanism, the water delivery mechanism, and the dispensing mechanism between the first chamber and the second chamber at timed intervals in order to continuously germinate a first batch of dosing material from water and a pellet in one chamber while simultaneously dispensing a second batch of dosing material from the other chamber.

2. The dual chamber bioremediation apparatus according to claim 1, wherein the dispensing mechanism further comprises a dispensing conduit attached to said container and a dosing spray member attached to the dispensing conduit adapted for spraying the dosing material into the waste drain for uniform distribution of the bioremediation bacteria in the waste drain.

3. A method for applying continuous bioremediation bacteria to a waste drain, comprising the steps of:
(a) preparing a first bioremediation dosing mixture in a first chamber;
(b) simultaneously dosing a drain line with active bioremediation bacteria mixture from a second chamber;
(c) dosing a drain line with the first bioremediation dosing mixture from the first chamber after exhaustion of the active bioremediation dosing mixture from the second chamber;
(d) simultaneously with step (c), preparing a second bioremediation dosing mixture of active bioremediation bacteria in the second chamber;
(e) alternately repeating steps (a)-(b) and (c)-(d) as the dosing mixture from the respective first and second chambers.

4. The method of claim 3 wherein the first bioremediation dosing mixture is prepared from a cartridge containing pellets of bioremediation bacteria.

5. The method of claim 3 wherein the first chamber contains a supply of water sufficient to provide a 24 hours supply of dosing mixture.

6. The method of claim 3 wherein said second chamber is prepared with a 24-hour supply of dosing mixture.

7. The method of claim 3 wherein the dosing mixture is added to the waste drain in predetermined time intervals.

8. The method of claim 7 wherein the time intervals comprise two hours.

9. The method of claim 3 wherein the supply of remediation bacteria is replenished once a month.

10. A bioremediation apparatus for producing and dosing high cell counts of bioremediation bacteria to a waste drain, comprising:
a replication chamber sufficient to contain at least a two-day supply of bioremediation bacteria dosing mixture;
a sensor to determine the amount of water in the replication chamber;
a pellet delivery mechanism positioned to periodically deliver at least one pellet containing a freeze-dried bacteria medium into the chamber;
a water delivery mechanism connected to fill the chamber;
a dispenser conduit mechanism attached to the chamber and adapted for connection to the waste drain;
at least one pump connected in line to the dispenser conduit mechanism to deliver bioremediation bacteria media to the waste drain; and
a powered control logic circuit programmed to: activate the pellet delivery mechanism to add a pellet to the chamber when about half of the volumn of the bioremediation media in the chamber has been dispensed; activate the water delivery system to fill the chamber; and activate the dispenser pump system at timed intervals to pass bioremedial bacteria into the waste drain.

11. The apparatus of claim 10 including a transformer, a water line, a pump and means for a before and after flush of the dispenser.

12. The apparatus of claim 10 including a transformer, a water line, a pump, and an air pump for introducing oxygen.

13. The apparatus of claim 10 including a transformer, a water line, a pump and a means for the simultaneous treatment of two waste drains.

14. The apparatus of claim 10 including a transfer, a water line, a pump, means for treating two waste drains, and an air pump for introducing oxygen.

15. The apparatus of claim 10 including a battery, a water line and a pump.

16. The apparatus of claim 10 including a battery, a water line and two pumps.

17. The apparatus of claim 10 including a transformer, a water reservoir and two pumps.

18. The apparatus of claim 10 including a transformer, a water reservoir, two pumps and an air pump for introducing oxygen.

19. The apparatus of claim 10 including a transformer, a water reservoir, two pumps, and means for the simultaneous treatment of two waste drains.

20. The apparatus of claim 10 including a transformer, a water reservoir, two pumps, and means for the simultaneous treatment of two waste drains, and an air pump.

21. The apparatus of claim 10 wherein the pellet delivery system comprises pellet wheel compartments and moisture barrier film, wherein pellets contained in said compartment are covered by said moisture barrier film.

22. The apparatus of claim 20 wherein said wheel is configured to rotate periodically to eject a pellet.

22. The apparatus of claim 21 containing means for rotating the wheel and individually removing the moisture barrier film from the pellets.

23. A method for producing enhanced replication of bioremedial bacteria in continuous waste drain treatment media, comprising:
a) preparing a chamber containing at least a two-day supply of dosing water;
b) feeding at least two pellets of bioremedial bacteria into the chamber to initiate replication;
c) withdrawing dosing media from the chamber in measured doses and timed stages while adding a single pellet to the chamber in daily stages;
d) refilling the chamber with water and a fresh pellet when the chamber is half empty;
e) withdrawing dosing media from the refilled chamber having a superior cell count of bioremedial bacteria for waste drain treatment; and
f) repeating steps (c), (d) and (e) to provide a continuous process.

24. The method of claim 23 wherein the bioremediation dosing mixture is prepared from a cartridge containing pellets of bioremediation bacteria.

25. The method of claim 23 wherein the chamber contains about five gallons of water.

26. The method of claim 23 comprising repeating steps (a) through (d) monthly to provide a continuous method.
